(19) United States
(12) Patent Application Publication
(10) Pub. No.: US 2013/0345257 A1
(43) Pub. Date: Dec. 26, 2013

(54) COMPOSITION FOR LUPUS NEPHRITIS AND METHODS OF MAKING AND USING THE SAME

(71) Applicant: The Regents of the University of California, Oakland, CA (US)

(72) Inventors: Bevra H. HAHN, Encino, CA (US); Maida Wong, Los Angeles, CA (US); Brian Skaggs, Encino, CA (US); Elaine Lourenco, Los Angeles, CA (US)

(73) Assignee: The Regents of the University of California, Oakland, CA (US)

(21) Appl. No.: 13/928,308

(22) Filed: Jun. 26, 2013

Related U.S. Application Data
(60) Provisional application No. 61/664,322, filed on Jun. 26, 2012.
(60) Provisional application No. 61/681,107, filed on Aug. 8, 2012.

(51) Int. Cl. A61K 31/4704

(52) U.S. Cl. CPC A61K 31/4704
USPC 514/312; 546/155

(57) ABSTRACT

Provided herein are compositions and methods associated with the prevention and treatment of lupus nephritis. In particular, provided herein is a method for delaying onset of active lupus nephritis in a subject at risk for developing active lupus nephritis, comprising periodically administering to the subject an amount of laquinimod effective to delay onset of active lupus nephritis in the subject. Also provided herein are laquinimod (LAQ) and a pharmaceutical composition comprising an amount of LAQ for use in delaying onset of active lupus nephritis in a subject at risk for developing active lupus nephritis.

### CROSS-REFERENCE TO RELATED APPLICATIONS

This application claims priority from U.S. Provisional Applications No. 61/664,322, filed on Jun. 26, 2012 and No. 61/681,107, filed on Aug. 8, 2012, each of which is hereby incorporated by reference herein in its entirety.

### FIELD

Provided herein are compositions for lupus nephritis treatment and prevention. Also provided herein are methods of making and using the same.

### BACKGROUND

Lupus nephritis (LN), characterized by inflammation of the kidney, is a complication which occurs in a subpopulation of patients with Systemic Lupus Erythematosus (SLE) and is one of the most serious complications caused by SLE. (MedlinePlus, 2010)

SLE is a debilitating autoimmune disease of great clinical diversity and can manifest itself in different ways and lead to a number of complications, e.g., arthritis, arthralgia, and myalgia, depending on the patient and the parts of the body affected. The precise etiology of SLE has not yet been determined, but hormonal, genetic, viral and environmental factors may precipitate the disease. SLE prevalence varies across ethnicities and geographic regions with an occurrence rate of 15 to 50 cases per 100,000 persons. SLE is most common in women of childbearing age (15-44) with a female-to-male ratio varying from 4.3 to 13.6 (Petri, 2002). Virtually all body systems may be involved, including the musculoskeletal, mucocutaneous, cardiovascular, neurological, respiratory, renal, ophthalmic hematological and gastrointestinal systems.

Due to the great clinical diversity and idiopathic nature of SLE, management of idiopathic SLE depends on its specific manifestations and severity (The Merck Manual, 2011). Therefore, medications suggested to treat SLE generally are not necessarily effective for the treatment of all manifestations of and complications resulting from SLE, e.g., LN.

LN usually arises early in the disease course, within 5 years of diagnosis. The pathogenesis of LN is believed to derive from deposition of immune complexes in the kidney glomeruli that initiates an inflammatory response (Brent, 2008).

An estimated 30-50% of patients with SLE develop nephritis that requires medical evaluation and treatment. LN is a progressive disease, running a course of clinical exacerbations and remissions. Early detection and treatment can significantly improve renal outcome and prognosis. Although over the last decades, treatment of LN has been greatly improved, 5 and 10-year survival rates are documented as 85% and 73%, respectively (Brent, 2008). LN morbidity is related to the renal disease itself, as well as to treatment-related complications.

Renal biopsy is considered for any patient with SLE who has clinical or laboratory evidence of active nephritis, in order to determine the histological type as well as the appropriate treatment management and prognosis. (Hahn, 2012; Brent, 2008)

The histological classification of LN was revised by the International Society of Pathology/Renal Pathology Society (ISN/RPS) in 2003 and is based on light microscopy, immunofluorescence, and electron microscopy findings from renal biopsy specimens (Foster, 2004). These classifications describes 6 major classes of LN: class I and II—mesangial LN, class III and IV—proliferative LN, class V—membranous LN and class VI—advanced sclerosis LN. The ISN/RPS classifications were based on earlier classifications by the World Health Organization (WHO) published in 1974 and 1982.

There is no existing cure for LN. The principal goals of therapy are to normalize renal function, urine sediment and proteinuria, reduce the frequency of relapses or prevent the progressive loss of renal function through mild, moderate and severe renal impairment to end stage renal disease (ESRD) requiring dialysis or kidney transplantation. Therapy varies pending on the histopathological findings as well as the clinical manifestations.

Corticosteroids and cytotoxic or immunosuppressive agents, particularly cyclophosphamide, azathioprine, or mycophenolate mofetil (MMF) are the standard of care for patients with aggressive proliferative LN, while less aggressive treatment options may be considered for purely membranous LN or mesangial LN. Angiotensin Converting Enzyme (ACE) inhibitors or Angiotensin II Receptor Blockers (ARBs) may control blood pressure and reduce proteinuria.

Most of the above mentioned treatments are not specifically indicated for the treatment of SLE/LN and treatment protocols vary.

Treatment of accompanying SLE signs, symptoms, and complications may additionally include a combination of Nonsteroidal anti-inflammatory drugs (NSAIDs), antimalarial agents, anti-hypertensives, calcium supplements or bisphosphonate, anti-coagulants and others.

While many patients fail to respond or respond only partially to the standard of care medications listed above, the long-term use of high doses of corticosteroids and cytotoxic therapies may have profound side effects such as bone marrow depression, increased infections with opportunistic organisms, irreversible ovarian failure, alopecia and increased risk of malignancy. Infectious complications coincident with active SLE and its treatment with immunosuppressive medications are the most common cause of death in patients with SLE.

What is needed in the art are more improved compositions and/or methods for using such in treating SLE, in particular, LN.

### SUMMARY

Provided herein is a method for delaying onset of active lupus nephritis in a subject at risk for developing active lupus nephritis, comprising periodically administering to the subject an amount of laquinimod effective to delay onset of active lupus nephritis in the subject.

Also provided herein are compositions comprising laquinimod for use in delaying onset of active lupus nephritis in a subject at risk for developing active lupus nephritis. Further provided herein are pharmaceutical compositions comprising an amount of laquinimod for use in delaying onset of active lupus nephritis in a subject at risk for developing active lupus nephritis.

In one aspect, provided herein is a method for delaying or preventing onset of active lupus nephritis in a mammal at risk for developing active lupus nephritis. The method comprises a step of periodically administering to the mammal an amount of laquinimod effective to delay onset of active lupus nephritis in the mammal. The following embodiments, when not mutually exclusive, can be combined in any way, cross different aspects of the invention described herein.

In some embodiments, the mammal is afflicted with class I lupus nephritis. In some embodiments, the mammal is afflicted with class II lupus nephritis. In some embodiments, the mammal's protein to creatinine ratio at baseline is less than 3. In some embodiments, wherein the mammal's protein to creatinine ratio at baseline is less than 2. In some embodiments, wherein the mammal's protein to creatinine ratio at baseline is less than 1. In some embodiments, wherein the laquinimod is a pharmaceutically acceptable salt of laquinimod. In some embodiments, the pharmaceutically acceptable salt of laquinimod is laquinimod sodium. In some embodiments, the periodic administration of laquinimod is effected orally.

In some embodiments, the amount of laquinimod is 0.25-2.0 mg/day. In some embodiments, the amount of laquinimod is 0.25 mg/day. In some embodiments, the amount of laquinimod is 0.3 mg/day. In some embodiments, the amount of laquinimod is 0.5 mg/day. In some embodiments, the amount of laquinimod is 1.5 mg/day. In some embodiments, the amount of laquinimod is 0.5-1.2 mg/day. In some embodiments, the amount of laquinimod is 0.6 mg/day. In some embodiments, the amount of laquinimod is 1.0 mg/day. In some embodiments, the amount of laquinimod is 1.2 mg/day.

In some embodiments, the amount of laquinimod is effective to prevent onset of active lupus nephritis in the mammal. In some embodiments, the amount of laquinimod is effective to delay or prevent a symptom of active lupus nephritis in the mammal. In some embodiments, the symptom is proteinuria in the mammal. In some embodiments, the symptom is increase of the mammal's protein to creatinine ratio. In some embodiments, symptom is increase of immune complex deposition in the mammal.

In some embodiments, the amount of laquinimod is effective to delay or prevent increase of glomerular immunoglobulin deposition in the mammal. In some embodiments, the amount of laquinimod is effective to delay or prevent increase of glomerular Complement component 3 (C3) deposition in the mammal. In some embodiments, the symptom is serum anti-DNA antibody production in the mammal. In some embodiments, the symptom is edema in the mammal. In some embodiments, the symptom is hypertension in the mammal.

In some embodiments, the amount of laquinimod is effective to reduce the mammal's protein to creatinine ratio. In some embodiments, the mammal's protein to creatinine ratio is reduced by at least 50% as compared to baseline. In some embodiments, the mammal's protein to creatinine ratio is reduced to no more than 0.3. In some embodiments, the periodic administration continues for at least 24 weeks. In some embodiments, the mammal is human. In some embodiments, laquinimod for use in delaying onset of active lupus nephritis in a mammal at risk for developing active lupus nephritis.

In one aspect, provided herein is a method for treating or alleviating a symptom associated with active lupus nephritis in a mammal diagnosed with active lupus nephritis. The method comprises a step of periodically administering to the mammal an amount of laquinimod effective to treat or alleviate a symptom associated with active lupus nephritis in the mammal. In some embodiments, the symptom is selected from a group consisting of elevated creatine level, proteinuria, hematuria, red blood cell casts, granular casts, microhematuria, macrohematuria, reduced renal function; rapidly progressive glomerulonephritis, acute renal failure, hyperkalemia; hypertension, tubular abnormalities; uremia due to retention of waste products and renal insufficiency such as azotemia (elevated blood nitrogen) and oliguria (low urine output<400 mL/day), a malar rash, a discoid rash, a photosensitivity, an oral ulcer, a nonerosive arthritis, a pleuropericarditis, and neurological manifestations, and hematological disorders.

In one aspect, provided herein is a pharmaceutical composition that comprises: an amount of laquinimod for use in delaying onset of active lupus nephritis in a mammal at risk for developing active lupus nephritis, and a pharmaceutical carrier or adjuvant. In one aspect, provided herein is a composition that comprises an active ingredient or compound which is effective for: induction of at least two types of regulatory cells that can suppress autoimmunity, and reduction of numbers of circulating moncytes/macrophages.

In some embodiments, the compound is Laquinimod (LAQ). In some embodiments, the compound is effective for lupus nephritis. In some embodiments, the regulatory cells express one or more markers selected from the group consisting of CD3, CD4, CD8, CD11, CD19, CD25, CD28, Foxp3, Tr1, Th3, CD8, CD28, Qa-1, CD11, Ly6G and Ly6C. In some embodiments, the composition further comprises a pharmaceutically acceptable carrier. In some embodiments, the composition is an oral formulation.

In one aspect, provided herein is a method of treating, ameliorating, or preventing lupus nephritis, comprising: applying to a mammal a composition according to any embodiment described herein.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts an exemplary embodiment: A) The development of proteinuria in mice between 27 and 32 weeks of age in each treatment group is shown (black line is controls treated with water). Five groups of mice were treated from 27 weeks. of age: 1) mice treated with water by gavage (H₂O): 2) mice treated with laquinimod at 1 mg/kg: LAQ1; 3) mice treated with laquinimod at 25 mg/kg: LAQ25 (Teva Pharmaceutical); 4) mice treated with mycophenolate mofetil at 30 mg/kg: MMF30, and 5) mice treated with mycophenolate mofetil at 100 mg/kg: MMF100. Note that both doses of LAQ as well as high dose MMF prevented the appearance of proteinuria. B) Plasma levels of creatinine (mean+/−SEM) are shown for the control and each treatment group at the time mice were 20 weeks old. All mice were treated prior to appearance of anti-dsDNA. Note that the rise4 in creatinine seen in the no treatment group did not occur in any of the treatment groups. Each group contains 7 to 11 mice. * indicates p<0.05.

FIG. 2 depicts an exemplary embodiment, illustrating that plasma levels of IgG anti-dsDNA, an autoantibody that causes lupus nephritis. Five groups of mice were treated from 10 wks. of age: 1) mice treated with water by gavage (H₂O): 2) mice treated with laquinimod at 1 mg/kg: LAQ1; 3) mice treated with laquinimod at 25 mg/kg: LAQ25 (Teva Pharmaceutical); 4) mice treated with mycophenolate mofetil at 30 mg/kg: MMF30, and 5) mice treated with mycophenolate mofetil at 100 mg/kg: MMF 100. ***indicates levels differ from those of water treated mice at p<0.001. ** indicates levels differ from those of water treated mice at p<0.01. *indicates levels differ from those of water treated mice at p<0.05. Note that mice receiving high dose MMF or either dose of LAQ had lower anti-DNA levels.

FIG. 3 depicts an exemplary embodiment, illustrating the preventive effects of low dose treatments. A) The percentages of mice that developed 2+ or greater proteinuria in each group at various ages are shown. All of these mice were treated prior to appearance of anti-dsDNA or proteinuria, starting at age 10-12 weeks with treatment up to 32 weeks of age. Five groups of mice were treated: 1) mice treated with water by gavage (H₂O): 2) mice treated with laquinimod at 1 mg/kg: LAQ1; 3) mice treated with laquinimod at 25 mg/kg: LAQ25 (Teva Pharmaceutical); 4) mice treated with mycophenolate mofetil at 30 mg/kg: MMF30, and 5) mice treated with mycophenolate mofetil at 100 mg/kg: MMF100. Note that proteinuria developed in water-treated controls by 30 weeks of age, but in none in the high dose MMF or either LAQ treatment group. B) shows the effects of low dose treatments beyond 32 weeks up until about 42 weeks. Percentages of mice developing proteinuria for mice that received water, treatment with LAQ 1 mg/kg per day 3 times a week (low dose: LAQ1) or MMF 30 mg/kg per day (low dose: MMF30) are compared. Note that both LAQ and MMF were effective in delaying onset of proteinuria. In particular, low dose LAQ treatment completely prevented onset of proteinuria in mice until 42th week. Each group contained 6 to 10 mice.

FIG. 4 depicts an exemplary embodiment, illustrating the effects of low dose treatment on mice survival. Survival results were compared for three groups of mice treated with water, LAQ 1 mg/kg 3 days a week or MMF 30 mg/kg, 5 days a week—all prior to appearance of anti-dsDNA and proteinuria. Note that mice in the control group began to die after 40 weeks, whereas mice in the treatment groups were still alive at 45 weeks (with the exception of one mouse in the MMF30 group. p value indicates the difference by ANOVA analysis.

FIG. 5 depicts an exemplary embodiment, illustrating the effects of LAQ in groups of mice with proteinuria (e.g., after development of clinical nephritis) and prior to development of proteinuria. In one set, mice were treated with either water or LAQ three days of each week prior to the development of proteinuria. Mice in the water-treated group began to develop proteinuria after 4 weeks. Mice in the LAQ treated group did not develop proteinuria six weeks after the treatment. This suggests that LAQ can prevent development of nephritis. In another set, mice that already developed proteinuria underwent the same treatment. Note that proteinuria among mice in the water-treated group was unaffected by water treatment (proteinuria continued in 100% of the mice). LAQ treated mice showed significant reduction of proteinuria. This suggests that LAQ can suppress established nephritis. Each group contained 6 to 10 mice.

FIG. 6 depicts an exemplary embodiment, further illustrating the effects of LAQ treatment on mice survival after proteinuria was present (e.g., after development of clinical nephritis). LAQ was administered at 25 mg/kg three times a week while control mice were only treated with water. Note that mice treated with water began to die after 39 weeks, whereas mice treated with LAQ survived.

FIG. 7 depicts an exemplary embodiment: A) illustrates gating technique used to identify CD4+ regulatory T cells (CD4+ Tregs), which are shown as the dotted cloud inside the box insert and also labeled as CD4+ Foxp3+. B) Percentages of CD4+ Tregs cells within the peripheral blood mononuclear (PBMC) population in the blood are shown for samples from untreated mice, mice treated with laquinimod at 1 mg/kg: LAQ (1 mg/kg), mice treated with laquinimod at 25 mg/kg: LAQ (25 mg/kg) (Teva Pharmaceutical); mice treated with mycophenolate mofetil at 30 mg/kg: MMF (30 mg/kg), mice treated with mycophenolate mofetil at 100 mg/kg: MMF (100 mg/kg). Note that Tregs are a significantly higher percent of cells in mice treated with either dose of LAQ compared to the other groups. **indicates p<0.01 compared to untreated controls. *** indicates p<0.001 compared to untreated controls.

FIG. 8 depicts an exemplary embodiment, illustrating the results of CD4 analysis in splenocytes. A) shows the level of CD4⁺ Tregs cells (also as CD4⁺CD25⁺Foxp3⁺ cells). B) shows the level of CD4⁺ cells. Note that percent of CD4⁺ Tregs increased only by the LAQ treatment (left panel). In contrast, mice treated with MMF have significantly lower percentage of CD4⁺ T cells, but LAQ-treated mice do not (right panel). Six to 11 mice are included in each group.

FIG. 9 depicts an exemplary embodiment, illustrating the results of CD8 analysis in splenocytes. In mice treated with LAQ, percentages of CD8⁺ cells in spleen are not significantly altered compared to the control mice. In mice treated with MMF, percentages of CD8⁺ cells in spleen are reduced compared to the control mice. Six to 11 mice are included in each group.

FIG. 10 depicts an exemplary embodiment, illustrating the results of various T cells. Results are shown for CD44^{hi}CD62L^{hi} in CD25⁺Foxp3⁺ (CD4⁺ Treg memory cells), CD44^{int}CD62L^{hi} in CD25⁺Foxp3⁺ (naïve CD4+ Treg cells), CD44^{hi}CD62L^{lo} in CD25⁺Foxp3⁺ (CD4⁺ Treg effector cells), and CD44^{hi}CD62L^{hi} in CD25⁺Foxp3⁺ (naïve CD4⁺ Treg cells). Six to 10 mice are included in each group.

FIG. 11 depicts an exemplary embodiment, illustrating histology analysis of kidneys. Histologic renal changes visualized by PAS staining of fixed kidney specimens are shown here. In mice treated with LAQ or MMF at the higher doses, inflammatory glomerular changes are compared with those of the water group, including hypercellularity, cellular crescents, hyaline deposition, neutrophilic infiltration, interstitial changes and total changes. Chronic scarring (irreversible) includes glomerular sclerosis (GS), focal segmental glomerular sclerosis (FSGS (, interstitial fibrosis and tubular atrophy (IFTA). Scarring is shown in the last two panels. Scarring was less in the groups treated with LAQ or MMF LAQ and MMF did not differ in these properties. Kidneys were harvested from mice treated before they had proteinuria, but followed until proteinuria developed in ½ of the controls. Histologic results from mice treated with water or with LAQ starting after proteinuria developed, are shown in the Tables 3 and 4. Although there is higher background scarring that was not completely reversed, there is significantly less histologic evidence of inflammation in the kidneys of mice treated with LAQ. Each group in all studies included 6 mice included per group and 50 glomeruli were analyzed.

FIG. 12 depicts an exemplary embodiment, illustrating the results of immunofluorescence analysis. Immunofluorescence on renal specimens from mice shown in FIG. 12 were studied on frozen specimens and analyzed for IgG deposition (A) and murine C3 complement deposition (B). Note that both of these were significantly decreased in mice treated prior to onset of clinical nephritis with LAQ or MMF.

FIG. 13 depicts an exemplary embodiment, illustrating that laquinimod (LAQ) prevents lupus nephritis in BWF1 mice. Young mice (12 weeks old) without established disease were treated orally with vehicle, laquinimod (1 or 25 mg/kg), or MMF (30 or 100 mg/kg) as described. (A) Survival, (B) proteinuria ≧300 mg/dL, (C) serum creatinine and (D) histology scores are reported. *p<0.05, **p<0.01, ***p<0.001, ns=p>0.05 treatment versus vehicle group. Values are expressed as mean±SEM and are representative of three independent experiments with similar results. GS, glomerulosclerosis; FSGS, focal segmental glomerulosclerosis; IFTA, interstitial fibrosis or tubular atrophy.

FIG. 14 depicts an exemplary embodiment, illustrating that LAQ ameliorates lupus nephritis in BWF1 mice with anti-dsDNA and low proteinuria (PU^{lo}). These mice are at an intermediate state of clinical activity; e.g., they have autoantibodies in serum but do not yet have heavy proteinuria from deposition of those antibodies in glomeruli. This state represents the time at which many patients with SLE present for medical care. Mice were treated orally with vehicle, laquinimod (25 mg/kg), or MMF (100 mg/kg) as described. (A) Survival, (B) proteinuria ≧300 mg/dL, (C) serum creatinine and (D) histology scores are reported. *p<0.05, **p<0.01, ***p<0.001, ns=p>0.05 treatment versus vehicle group. Values are expressed as mean±SEM and are representative of four independent experiments with similar results. GS, glomerulosclerosis; FSGS, focal segmental glomerulosclerosis; IFTA, interstitial fibrosis or tubular atrophy.

FIG. 15 depicts an exemplary embodiment, illustrating that LAQ ameliorates lupus nephritis in BWF1 mice with high proteinuria (PU^{hi}). Here, the mammal undergoing treatment already have established, active lupus nephritis that is threatening to produce renal failure. Mice were treated orally with vehicle, laquinimod (25 mg/kg) 3 times a week, or MMF (100 mg/kg) 5 times a week as described. (A) Survival, (B) proteinuria <100 mg/dL, (C) serum creatinine and (D) histology scores are reported. *p<0.05, **p<0.01, ***p<0.001, ns=p>0.05 treatment versus vehicle group. Values are expressed as mean±SEM and are representative of four independent experiments with similar results. GS, glomerulosclerosis; FSGS, focal segmental glomerulosclerosis; IFTA, interstitial fibrosis or tubular atrophy. Note that LAQ treatment at this time suppresses proteinuria and rise in serum creatinine, indicating that renal function is preserved, or at least does not deteriorate as it does in water-treated controls.

FIG. 16 depicts an exemplary embodiment, illustrating that induction of myeloid-derived suppressor cells (MDSC) and inhibition of M/M after laquinimod treatment. BWF1 were treated with laquinimod (25 mg/kg), MMF (100 mg/kg) or water (vehicle) either before (Prev) or after anti-dsDNA onset but low proteinuria)(PU^{lo}. Cells from spleen and kidney were investigated by FACS at 25 (Prev) or 10 (PU^{lo}) weeks after treatment was initiated. Graphs compare percentages of (A) CD11b⁺Ly6C⁺Ly6G⁺ cells, (B) CD11b⁺Ly6C⁺Ly6G⁻ cells, and (C) monocyte/macrophages (CD11b⁺Ly6C⁻Ly6G⁻). (D) Inhibition of CFSE-labeled CD3/28-stimulated CD4⁺CD25⁻ T cell proliferation by gr-MDSC(CD11b⁺Ly6C⁺Ly6G⁺ cells) or mo-MDSC(CD11b⁺Ly6C⁺Ly6G⁻ cells) sorted from spleens of laquinimod-, MMF- or vehicle-treated mice (ratio 1:1). Values are expressed as mean±SEM and are representative of three independent experiments with similar results. (*p<0.05, ** p<0.01, ***p<0.001-one-way ANOVA). A novel aspect of this experiment is that cells attacking the target organ (i.e. kidneys) were isolated and characterized. Therefore, we can state that LAQ treatment suppresses numbers of monocytes/macrophages (known to mediate fibrosis) in the target tissue, and it induces regulatory myeloid-derived suppressor cells that can suppress inflammation in the target tissue. The LAQ treatment has direct effects on cells in the target organ that result in some protection of that organ from inflammation and damage.

FIG. 17 depicts an exemplary embodiment, illustrating that LAQ promotes a cytokine shift to an anti-inflammatory profile and down-regulates activation/co=stimulatory molecules. Spleens were removed 7 weeks after initiation of laquinimod treatment in PU^{lo} animals. Splenocytes were stimulated with TLR4 (LPS), 7 (imiquimod), or 9 (ODN) agonists, or PMA plus ionomycin, as described. FACS plots are representative of one experiment showing intracellular production of (A) IL-10 or (B) TNFα by monocyte/macrophages (CD11b+Ly6C−Ly6G−), and (C) IFNγ by CD4⁺ T cells from laquinimod- or vehicle-treated mice. Graphs of IL-10, TNFα, and IFNγ production represent mean±SEM of cytokine production (n=6/gp, *p<0.05, **p<0.01, ***p<0.001 using two way ANOVA). (D) Ex vivo and in vitro (1 ug/mL LPS for 24 h) expression of activation/costimulatory molecules on monocyte/macrophages isolated from the same splenocytes is down-regulated by laquinimod treatment. Graphs depict mean±SD of mean fluorescence intensity (MFI) and are from three independent experiments with similar results. (n=4/gp, *p<0.05, **p<0.01, ***p<0.001 using t test).

FIG. 18 depicts an exemplary embodiment, illustrating that LAQ and MMF delay glomerular damage and IgG/C3 deposition. Kidney sections were stained with H&E (A-C) or fluorescently stained with FITC-labeled anti-mouse IgG (D-F) or anti-mouse C3 (G-I) antibodies. Pictures are representative of multiple kidney sections from multiple mice in each treatment group.

### DETAILED DESCRIPTION

### Definitions

As used herein, and unless stated otherwise, each of the following terms shall have the definition set forth below. Further, unless otherwise noted, additional terms are to be understood according to conventional usage by those of ordinary skill in the relevant art.

As used herein, an “amount” or “dose” of laquinimod as measured in milligrams refers to the milligrams of laquinimod acid present in a preparation, regardless of the form of the preparation. Therefore, a “dose of 0.5 mg laquinimod” means the amount of laquinimod acid in a preparation is 0.5 mg, regardless of the form of the preparation. Thus, when in the form of a salt, e.g. a laquinimod sodium salt, the weight of the salt form necessary to provide a dose of 0.5 mg laquinimod would be greater than 0.5 mg due to the presence of the additional salt ion.

As used herein, “laquinimod” means laquinimod acid, a homolog, or a pharmaceutically acceptable salt thereof.

As used herein, a subject “afflicted with Systemic Lupus Erythematosus (SLE)” means a subject who has been clinically diagnosed to have Systemic Lupus Erythematosus. Subject and mammal are used interchangeably.

As used herein, a subject afflicted with “active lupus nephritis” means a subject who has been clinically diagnosed to have active lupus nephritis based on International Society of Nephrology/Renal Pathology Society (ISN/RPS 2003) classification of lupus nephritis—classes III (A or A/C), IV-S or IV-G (A or A/C), or class V—pure or in combination with class III or IV. In addition, clinically active LN is evident by protein to creatinine ratio >0.5, for LN class III, IV or [class V in combination with class III or IV] or protein to creatinine ratio >1 for LN class V. “Active lupus nephritis” as used herein specifically excludes class I lupus nephritis and class II lupus nephritis.

Proteinuria, or the presence of excess serum proteins in the urine, is one of the key indicators of the onset of renal involvement in SLE, protein to creatinine ratio in the urine is a reliable measure of proteinuria in patients with lupus nephritis (Christopher-Stine, 2004). A high protein to creatinine ratio may be indicative of renal disease, such as lupus nephritis.

As used herein, a subject who is “at risk for developing active lupus nephritis” is afflicted with SLE or class I or class II lupus nephritis. A subject who is “at risk for developing active lupus nephritis” is not afflicted with active lupus nephritis.

As used herein, a subject “afflicted with class I lupus nephritis” is a subject who has minimal mesangial lupus nephritis defined as class I in ISN/RPS 2003 classification of lupus nephritis. Similarly, a subject “afflicted with class II lupus nephritis” is a subject who has mesangial proliferative lupus nephritis defined as class II in the ISN/RPS 2003 classification of lupus nephritis.

As used herein, “delaying onset of active lupus nephritis” in a subject who is “at risk for developing active lupus nephritis” means prolonging the time to or preventing the progression of the Systemic Lupus Erythematosus, class I or class II lupus nephritis to active lupus nephritis.

As used herein, a subject at “baseline” is as subject prior to administration of laquinimod.

As used herein, “effective” when referring to an amount of laquinimod refers to the quantity of laquinimod that is sufficient to yield a stated therapeutic response without undue adverse side effects (such as toxicity, irritation, or allergic response) commensurate with a reasonable benefit/risk ratio when used in the manner of this invention.

As used herein, a “symptom” associated with active lupus nephritis includes any clinical or laboratory manifestation associated with lupus nephritis and is not limited to what the subject can feel or observe. For example, proteinuria is a symptom of active lupus nephritis.

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

Lupus Nephritis

Medical treatments for lupus nephritis (LN) depend on the severity of the disease in a patient. For example, for mild cases of the disease, corticosteroids are, in general, prescribed. More severe cases of the disease require treatments with immunosuppressant agents since LN depends on autoAb deposition and activation of multiple cell types that infiltrate kidneys and promote inflammation, including e.g., monocytes/macrophages (MM), DCs, T and B cells.

The two most commonly used agents are mycophenolate mofetil and intravenous cyclophosphamide. Both agents are associated with significant adverse effects: cyclophosphamide may induce permanent infertility in young women, and mycophenolate mofetil is associated with a higher risk of infection-related death. See, for example, Appel G B, et al. (2009). “Oral Mycophenolate Mofetil is not Superior to Intravenous Cyclophosphamide as Induction Therapy for Lupus Nephritis,” J Am Soc Nephrol 20 (5): 1103-1112.

In some cases where lupus-related thrombotic thrombocytopenic purpura is present, plasmapheresis is life-saving, and must be instituted early to avoid a poor outcome. See, for example, Chak, W K; et al., 2003, “Thrombotic thrombocytopenic purpura as a rare complication in childhood Systemic Lupus Erythematosus: case report and literature review,” Hong Kong Med J 9 (5): 363-368, which is hereby incorporated by reference herein in its entirety.

Immunomodulatory Compositions as Therapeutic or Prophylactic Agents

In one aspect, an Immunomodulatory agent is used to treat or prevent lupus nephritis. In some embodiments, the Immunomodulatory agent is laquinimod (LAQ). Other immunomodulatory agents used to treat lupus nephritis include antimalarials (particularly hydroxychloroquine which is the most widely used in the United States) and the biologic anti-B cell agent, rituximab. Immunosuppressive treatments include glucocorticoids, cyclophosphamide, mycophenolate mofetil, myphortic acid, azathioprine, cyclosporine and tacrolimus. Among these the FDA has approved use for treatment of lupus nephritis only glucocorticoids and hydroxychloroquine. The anti-B cells biologic belimumab has been approved for treatment of SLE, but not for lupus nephritis.

In some embodiments, laquinimod is a crystalline form of laquinimod, a laquinimod homolog, a laquinimod salt, laquinimod sodium, an amorphous form of laquinimod sodium, a polymorphic form of laquinimod sodium,

Any homologs or derivatives of laquinimod can be used in the compositions and methods provided herein. Exemplary salts and derivatives of laquinimod can be found, for example, in U.S. Pat. No. 6,077,851; No. 6,875,869; each of which is hereby incorporated by reference herein in its entirety.

In some embodiments, treatments are administered orally, as tablets, capsules, powder or a solution, to a mammal in need of such treatment. In some embodiments, treatments are achieved by parenteral administration through intramuscular, intravenous, subcutaneous or intrathecal injection, or infusion, to a mammal in need of such treatment. In some embodiments, the mammal is a human. In some embodiments, the mammal includes but is not limited to a dog, a mouse, a rat, a cow, a sheep, a goat, or a monkey.

In some embodiments, laquinimod (LAQ) exhibits many effects equivalent or superior to Mycophenolate Mofetil (MMF) or mycophenolate (standard care in human lupus nephritis) in treatment of lupus nephritis.

Laquinimod differs from mycophenolate in three ways: a) induction of at least two types of regulatory cells that can suppress autoimmunity; b) reduction of numbers of circulating and intrarenal monocytes/macrophages; and c) switch of renal macrophage phenotype from pro-inflammatory M1 to protective M2. These features could offer two clinical advantages over mycophenolate: a) fewer clinical flares of lupus nephritis over time in patients treated with laquinimod compared to those treated with mycophenolate and b) less renal damage over time in patients treated with laquinimod, since damage depends in part on activated macrophages within renal tissue.

Laquinimod (LAQ) administered to human beings down-regulates antigen (Ag) presentation, decreases chemokine production, decreases MHC expression on MM, and induces apoptotic pathways in PBMC (Gurevich M et al 2010). LAQ reduces progression of relapsing remitting multiple sclerosis (Comi G et al NEJM 2012); results of an early phase II trial in human lupus nephritis suggest it may be useful for suppression of clinical nephritis (Jayne D et al., presented at European League against Rheumatism Annual Meeting June 2013). MMF targets primarily lymphocytes; it is effective in many LN patients.

LAQ is an Immunomodulatory drug (not currently approved by FDA for any indication) which has been effective in reducing progression of relapsing remitting relapsing multiple sclerosis in humans, and in suppressing EAE in rodents. LAQ has several immunomodulatory actions and down-regulates activation of lymphocytes and monocyte-macrophages. In some embodiments, NZB×NZW(F1) female (BWF1) mice are used to demonstrate that Laquinimod can suppress lupus nephritis. Since the current treatment of lupus nephritis is primarily a combination of prednisone plus mycophenolate mofetil (MMF), results from the two agents (LAQ and MMF) are compared. Both agents are administered orally. In some embodiments, groups of 9-12 young (10-week-old) BWF1 females, negative for anti-DNA and proteinuria, are treated by oral gavage a plurality of times each week, for example, two or more times, three or more times, four or more times, five or more times, six or more times, seven or more times.

In some embodiments, a lower dose of the agent is used for a prophylactic treatment in comparison to a therapeutic treatment.

In some embodiments, an agent is administered at a dose proportional to the body mass of the subject receiving the agent, ranging from 0.001 mg/kg to over 1000 mg/kg per dose. In some embodiments, the agent is administered at a dose of 0.001 to 0.01 mg/kg or more, 0.01 to 0.1 mg/kg or more, 0.1 to 0.5 mg/kg or more, 0.5 to 1 mg/kg or more, 1 to 5 mg/kg or more, 5 to 10 mg/kg or more, 10 to 25 mg/kg or more, 25 to 50 mg/kg or more, 50 to 100 mg/kg or more, 100 to 150 mg/kg or more, 150 to 200 mg/kg or more, 200 to 250 mg/kg or more, 250 to 300 mg/kg or more, 300 to 500 mg/kg or more, 500 to 1000 mg/kg or more.

In some embodiments, an agent is administered to a subject one time a day. In some embodiments, an agent is administered to a subject 2 or 3 times a day. In some embodiments, an agent is administered to a subject 4 or more times a day. In some embodiments, an agent is administered to a subject once a week. In some embodiments, an agent is administered to a subject 2 or 3 times a week. In some embodiments, an agent is administered to a subject 4 or more times a week.

In some embodiment, the agent is administered continuously, e.g., at a low dose, over an extended period time, using a patch, an implant, or a portable drug dispensing pump.

In some embodiments, the subject is treated for an extended period of time, such as a month a longer, two months or longer, three months or longer, four months or longer, five months or longer, six months or longer, seven months or longer, eight months or longer, nine months or longer, 10 months or longer, 11 months or longer, 12 months or longer, 16 months or longer, 20 months or longer, 24 months or longer, 30 months or longer, 36 months or longer.

A significant reduction of new MRI lesions in the brains of patients with RRMS treated with LAQ compared to placebo was reported (Comi G. et al. 2012, “Oral laquinimod for multiple sclerosis,” N Engl J Med 366:1000-1009). The immunomodulatory effects of LAQ in humans has been reported as alteration of gene expression in PBMC (Gurevich M. et al. 2010, “Laquinimod suppresses antigen presentation in relapsing-remitting multiple sclerosis: In vitro high-throughput gene expression study,” J Neuroimmunol 221:87-94) and as induction of myeloid suppressor cells in rodent EAE (Schulze-Topphoff U. et al. 2012, Laquinimod, a quinoline-3-carboxamide, induces type II myeloid cells that modulate CNS autoimmunity. PLoS One; e33797 Epub 2012 Mar. 30).

Recent studies have shown the importance of activated tissue and circulating monocyte/macrophages (M/M) in mediating damage to renal tissue in BWF1 mice and in people with lupus nephritis (Schiffer L. et al. 2008; “Activated renal macrophages are markers of disease onset and disease remission in lupus nephritis,” J Immunol 180:1938-1947).

Because LAQ has the potential to reduce activation of M/M and to switch tissue macrophages from M1 toward M2 phenotypes, it can have more prolonged benefit than treatment with agents like MMF that primarily deplete and inactivate lymphocytes. MMF is now standard in the care of patients with severe lupus nephritis (Hahn B H et al. 2012, American College of Rheumatology guidelines for screening, treatment and management of lupus nephritis. Arthritis Care Res (Hoboken) 64:797-808; and Dooley M A et al. 2011, “Mycophenolate versus azathioprine as maintenance therapy for lupus nephritis,” N Engl J Med 365:1886-1895). In some embodiments, MMF is used as a bench compound against which a potential therapeutic agent is compared. Although short-term renal disease is suppressed by prednisone plus MMF or cyclophosphamide, end-stage renal disease still occurs in a substantial portion of patients, especially African Americans (Costenbader K H et al. 2011, “Trends in the incidence, demographics, and outcomes of end-stage renal disease due to lupus nephritis in the US from 1995 to 2006,” Arthritis Rheum 63:1681-1688). In the short-term studies done by us with LAQ, clinical benefits of MMF and LAQ were similar, but suppression of M/M by LAQ will result in less renal damage over the long term.

Method of Making

A compound disclosed can be readily prepared according to established methodology in the art of organic synthesis. General methods of synthesizing the compound can be found in, e.g., Stuart Warren and Paul Wyatt, Workbook for Organic Synthesis: The Disconnection Approach, second Edition, Wiley, 2010. Synthesis of the compound is exemplified in Examples where the preparation of more than 41 different compounds is described in detail.

Methods of forming the composition generally comprise providing a compound disclosed herein and forming a composition comprising the compound. Processes and methods for the preparing laquinimod and salts and derivatives thereof can be found, for example, in U.S. Pat. No. 6,077,851; No. 6,875,869; each of which is hereby incorporated by reference herein in its entirety.

Method of Use for Prophylactic and Therapeutic Treatments

Lupus nephritis is one of the most serious complications caused by SLE. Therefore, there is a need for therapies for delaying or preventing disease progression from SLE to active lupus nephritis.

In some embodiments, compositions/methods provided herein are effective for treating active lupus nephritis, a disorder or a symptom related therewith in a subject, for example, a mammal such as a human. In some embodiments, compositions/methods provided herein are effective for alleviating a disorder or one or more symptoms associated with lupus nephritis in a subject, for example, a mammal such as a human. Generally, the method comprises applying to a subject a composition disclosed herein.

Exemplary disorder or symptoms include but are not limited to proteinuria (e.g., small amounts of protein are lost in the urine); hematuria (blood in the urine with red blood cell (RBC) casts present in the urine nephrotic syndrome, including granular casts, red cell casts, microhematuria, macrohematuria); reduced renal function; rapidly progressive glomerulonephritis (RPGN), acute renal failure (ARF), hypertension; hyperkalemia; hypertension (high blood pressure)—mild; tubular abnormalities; uremia (renal failure) due to retention of waste products and renal insufficiency such as azotemia (elevated blood nitrogen) and oliguria (low urine output <400 mL/day). Additional exemplary symptoms include a malar rash, a discoid rash, a photosensitivity, an oral ulcer, a nonerosive arthritis, a pleuropericarditis, a renal disorder, neurological manifestations, and hematological disorders.

In one embodiment, the subject is afflicted with class I lupus nephritis. In another embodiment, the subject is afflicted with class II lupus nephritis. In one embodiment, the subject is afflicted with class III lupus nephritis. In another embodiment, the subject is afflicted with class VI lupus nephritis. In one embodiment, the subject is afflicted with class V lupus nephritis. In another embodiment, the subject is afflicted with class VI lupus nephritis.

In some embodiments, provided herein is a method for delaying or preventing onset of active lupus nephritis in a subject, for example, a mammal such as a human at risk for developing active lupus nephritis, comprising periodically administering to the subject an amount of laquinimod effective to delay onset of active lupus nephritis in the subject.

In some embodiments, the subject's risk for developing active lupus nephritis is assessed by genetic profiling analysis, based on, for example, DNA microarray analysis of samples from patients suspected of genetic predisposition of developing SLE and/or LN. Exemplary markers associated with a risk developing active lupus nephritis in a subject such as a mammal like a human include but are not limited to TGFB1, IRF5, STAT4 genes and TRAF1-C allelic variants. More details can be found, for example, in Tsao B P 1998, Genetic susceptibility to lupus nephritis, Lupus 7(9):585-590; Vuong et al., 2010 Genetic Risk Factors in Lupus Nephritis and IgA Nephropathy—No Support of an Overlap, PloS One, volume 5 (5): e10559, each of which is hereby incorporated by reference in its entirety.

In some embodiments, the amount of laquinimod is effective to prevent onset of active lupus nephritis in a subject, for example, a mammal such as a human. In some embodiments, a lower amount of laquinimod is used to prevent onset of active lupus nephritis in a subject when compare to an amount used in a typical therapeutic treatment. For example, 90% or less, 80% or less, 70% or less, 60% or less, 50% or less, 40% or less, 30% or less, 20% or less, 15% or less, 12% or less, 10% or less, 9% or less, 8% or less, 7% or less, 6% or less, 5% or less, 4% or less, 3% or less, 2% or less, 1% or less, 0.5% or less, 0.2% or less, or 0.1% or less of the an amount of laquinimod used in a typical therapeutic treatment is used. In some embodiments, 1 mg/kg of laquinimod is used in a prophylactic treatment in a subject without active lupus nephritis. In some embodiments, 25 mg/kg of laquinimod is used in a therapeutic treatment of active lupus nephritis in a subject.

In some embodiments, the amount of laquinimod is effective to delay, alleviate, or prevent a symptom of active lupus nephritis in the subject. In some embodiments, the amount of laquinimod is effective to delay, alleviate, or prevent more than one symptoms of active lupus nephritis in the subject. In one embodiment, the symptom is proteinuria in the subject. In another embodiment, the symptom is increase of the subject's protein to creatinine ratio. In another embodiment, the symptom is increase of immune complex deposition in the subject. In another embodiment, the amount of laquinimod is effective to delay or prevent increase of glomerular immunoglobulin deposition in the subject. In another embodiment, the amount of laquinimod is effective to delay or prevent increase of glomerular Complement component 3 (C3) deposition in the subject. In another embodiment, the symptom is serum anti-DNA antibody production in the subject. In another embodiment, the symptom is edema in the subject. In yet another embodiment, the symptom is hypertension in the subject.

In one embodiment, the amount of laquinimod or is effective to reduce the subject's protein to creatinine ratio. In another embodiment, the subject's protein to creatinine ratio is reduced by at least 50% as compared to baseline. In yet another embodiment, the subject's protein to creatinine ratio is reduced to no more than 0.3.

In one embodiment, the periodic administration continues for at least 24 weeks. In another embodiment, the subject is human.

This invention also provides laquinimod for use in delaying onset of active lupus nephritis in a subject at risk for developing active lupus nephritis.

This invention also provides a pharmaceutical composition comprising an amount of laquinimod for use in delaying onset of active lupus nephritis in a subject at risk for developing active lupus nephritis.

For the foregoing embodiments, each embodiment disclosed herein is contemplated as being applicable to each of the other disclosed embodiment.

It is understood that where a parameter range is provided, all integers within that range, and hundredth thereof, are also provided by the invention. For example, “0.25-2000.0 mg/day” includes 0.25 mg/day, 0.26 mg/day, 0.27 mg/day, etc., up to 2000.0 mg/day. In one embodiment, the subject's protein to creatinine ratio at baseline is less than 3. In another embodiment, the subject's protein to creatinine ratio at baseline is less than 2. In another embodiment, the subject's protein to creatinine ratio at baseline is less than 1.

In one embodiment, the subject is female. In another embodiment, the subject is between 15-44 years of age. In another embodiment, the subject is a female of child-bearing age. In another embodiment, the subject is of Asian ethnicity. In another embodiment, the subject is of African ethnicity. In another embodiment, the subject is of Caucasian ethnicity. In another embodiment, the subject is Hispanic. In another embodiment, the subject is genetically predisposed to SLE. In another embodiment, the subject is genetically predisposed to LN. In another embodiment, the subject does not have vascular lesions. In another embodiment, the subject does not have glomerular lesions. In another embodiment, the subject does not have tubulointerstitial lesions. In another embodiment, the subject is afflicted one or more of arthralgias, arthritis, myalgia, adenopathy, malar rash, skin lesion, skin rash, discoid rash, photosensitivity, oral ulcers, serositis, leucopenia, lymphopenia, hemolytic anemia, thrombocytopenia, neurologic disorder, pleuritis, pericarditis, Central Nervous System (CNS) inflammation, cognitive impairment, systemic sclerosis, rheumatoid-like polyarthritis, polymyositis, dermatomyositis, hematologic cytopenia, positive test for anti-DMA, anti-Smith, or antiphospholipid antibodies, and antinuclear antibodies in high titers.

In one embodiment, the laquinimod is a pharmaceutically acceptable salt of laquinimod. In another embodiment, the pharmaceutically acceptable salt of laquinimod is laquinimod sodium. In yet another embodiment, the periodic administration of laquinimod is effected orally.

In one embodiment, the amount of laquinimod administered is 25 mg/kg 3 to 5 times a week. In another embodiment, the total amount of laquinimod administered is 0.25-2000.0 mg per day. In another embodiment, the total amount of laquinimod administered is 0.25 mg/day. In another embodiment, the total amount of laquinimod administered is 0.3 mg per day. In another embodiment, the total amount of laquinimod administered is 1.5 mg per day. In another embodiment, the total amount of laquinimod administered is 0.5-1.2 mg per day. In another embodiment, the total amount of laquinimod administered is 0.6 mg or more per day. In another embodiment, the total amount of laquinimod administered is 1.0 mg or more per day. In yet another embodiment, the total amount of laquinimod administered is 1.2 mg or more per day. In yet another embodiment, the total amount of laquinimod administered is 1.5 mg or more per day. In yet another embodiment, the total amount of laquinimod administered is 2 mg or more per day. In yet another embodiment, the total amount of laquinimod administered is 2.5 mg or more per day. In yet another embodiment, the total amount of laquinimod administered is 5 mg or more per day. In yet another embodiment, the total amount of laquinimod administered is 10 mg or more, 15 mg or more, 20 mg or more, 25 mg or more, 30 mg or more, 35 mg or more, 40 mg or more, 45 mg or more, 50 mg or more, 55 mg or more, 60 mg or more, 65 mg or more, 70 mg or more, 75 mg or more, 80 mg or more, 85 mg or more, 90 mg or more, 95 mg or more, 100 mg or more, 110 mg or more, 120 mg or more, 130 mg or more, 140 mg or more, 150 mg or more, 160 mg or more, 170 mg or more, 180 mg or more, 190 mg or more, 200 mg or more, 250 mg or more, 300 mg or more, 400 mg or more, 500 mg or more, 600 mg or more, 700 mg or more, 800 mg or more, 1000 mg or more, or 2000 mg or more per day.

In one embodiment, the subject at baseline has been treated with corticosteroids. In another embodiment, the subject at baseline has been treated with immunosuppressants. In another embodiment, the subject at baseline has been treated with cytotoxic agents. In another embodiment, the subject at baseline has been treated with ACE inhibitors or ARBs. In another embodiment, the subject at baseline has been treated with NSAIDs, antimalarial agents, anti-hypertensives, calcium supplements, bisphosphonate or anti-coagulants.

Pharmaceutical Compositions

In some embodiments, compositions provided herein include a pharmaceutical composition. In some embodiments, a pharmaceutical composition provided herein can optionally include a pharmaceutically acceptable carrier. The pharmaceutical composition may contain, as active ingredients, the aforementioned compound and optionally other compounds, or may contain a mixture of two or more aforementioned compounds.

The pharmacologically acceptable salt in the present specification is not specifically limited as far as it can be used in medicaments. Examples of a salt that the compound of the present invention forms with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

Further, the compounds of the present invention include hydrates thereof, various pharmaceutically acceptable solvates thereof, and polymorphic crystals thereof.

In some embodiment, the composition is administered continuously, e.g., at a low dose, over an extended period time, using a patch, an implant, or a portable drug dispensing pump.

A preferred formulation of the composition is oral formulation.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Patent Application Publication No. 2005/0192315 and PCT International Application Publication No. WO 2005/074899, each of which is hereby incorporated by reference herein in its entirety.

A dosage unit may comprise a single compound or mixtures of compounds thereof. A dosage unit can be prepared for oral dosage forms, such as tablets, capsules, pills, powders, and granules.

In some embodiments, laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit is preferably in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Patent Application Publication No. 2005/0192315, PCT International Application Publication Nos. WO 2005/074899, WO 2007/047863, and WO 2007/146248, each of which is hereby incorporated by reference into this application in its entirety.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwooci Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

The use of laquinimod for SLE had been previously suggested in, e.g., U.S. Pat. No. 6,077,851. However, the '851 patent does not disclose the use of laquinimod for the particular sub-population of SLE relevant to the instant invention. That is, the '851 patent does not disclose the use of laquinimod for subjects who are at risk for developing active lupus nephritis.

The use of laquinimod for treating patient afflicted with active lupus nephritis had been previously suggested in U.S. Patent Application Publication No. 2011/0218179 A1. However, the '179 publication also does not disclose the use of laquinimod for the particular sub-population of subjects that have not yet developed, but are at risk for developing active lupus nephritis.

On the other hand, laquinimod is surprisingly effective in delaying or preventing active lupus nephritis in subjects have not yet developed, but are at risk for developing active lupus nephritis.

### Markers and Assays for Ascertaining Renal Functionalities and Treatment Effects

In one aspect, effects of the treatments disclosed herein in a patient are ascertained by quantifying or assaying for markers or symptoms associated with systemic lupus erythematosus, in particular, lupus nephritis. Exemplary markers associated with a risk of developing active lupus nephritis in a subject such as a mammal like a human include but are not limited to TGFB1, IRF5, STAT4 genes and TRAF1-C % locus.

Additional examples of markers include biomarkers that correlate with SLE renal activity in longitudinal studies such as Chemokines, Neutrophil Gelatinase-Associated Lipocalin (NGAL), Tumor Necrosis Factor-Like Inducer of Apoptosis (TWEAK), Urine Proteomics markers, Autoantibodies such as Anti-C1q Antibodies, Antinucleosome Antibodies, and Anti-α-Actinin Antibodies; biomarkers that correlate with lupus nephritis activity in cross-sectional studies such as MAGE-B2 antibodies, Anti-CRP antibody, Serum and urine IL-12, Peripheral blood leukocyte chemokine transcriptional levels, Serum apoCIII, Serum ICAM-1, Antiendothelial cell antibody, Urine osteoprotegerin (OPG), FOXP3 mRNA expression in urinary sediments, Urine endothelin-1, Urine CXCR3+CD4+T cells, Serum and urine IL-12, Urine VCAM-1, P-selectin, TNFR-1, and CXCL16, Urine TGFβ-1, TGFβ and MCP-1 mRNA expression in urine sediments; biomarkers that correlate with renal histology of lupus nephritis such as Serum nitrate and nitrite levels, Glomerular MCP-1 expression, β1-integrin (CD29) expression on T cells, Chemokine and growth factor mRNA levels in urinary sediments, Antiribosomal P antibody and Urine glycoprotein panel; biomarkers that correlate with prognosis of lupus nephritis such as mEPCR expression on renal biopsy, VEGF expression, Glomerular MCP-1 expression, Serum nitrate and nitrite levels, STAT-1 expression on renal biopsy and Chemokine and growth factor mRNA levels in urinary sediments. Additional information can be found in, for example, Chi Chiu Mok, 2010, “Biomarkers for Lupus Nephritis: A Critical Appraisal,” Journal of Biomedicine and Biotechnology, Article ID 638413, 11 pages, which is incorporated by reference herein in its entirety.

Lupus nephritis is an inflammation of the kidney caused by systemic lupus erythematosus (SLE), a disease of the immune system. General symptoms of lupus include malar rash, discoid rash, photosensitivity, oral ulcers, non-erosive arthritis, pleuropericarditis, renal disease, neurological manifestations, and hematological disorders. Apart from the kidneys, SLE can also damage the skin, joints, nervous system and virtually any organ or system in the body. About half of cases of SLE demonstrate signs of lupus nephritis at one time or another. Renal-specific indications include proteinuria (e.g., small amounts of protein are lost in the urine); hematuria (blood in the urine with red blood cell (RBC) casts present in the urine, including granular casts, red cell casts, microhematuria, macrohematuria); reduced renal function; rapidly progressive glomerulonephritis (RPGN), acute renal failure (ARF), hypertension; hyperkalemia; hypertension (high blood pressure)—mild; tubular abnormalities; uremia (renal failure) due to retention of waste products and renal insufficiency such as azotemia (elevated blood nitrogen) and oliguria (low urine output <400 mL/day).

In some cases, micrographs of diffuse proliferative lupus nephritis have revealed increased mesangial matrix and mesangial hypercellularity.

In particular, the main clinical features are hypertension and RBC casts. The proteinuria in nephritic syndrome is not usually severe, but may occasionally be heavy enough to be in the range usually found in nephrotic syndrome.

In some embodiments, one or more markers are measured to assess the effects of a composition provided herein. In some embodiments, two or more markers are measured to assess the effects of a composition provided herein. In some embodiments, three or more markers are measured to assess the effects of a composition provided herein. In some embodiments, three or more markers are measured to assess the effects of a composition provided herein. In some embodiments, four or more markers are measured to assess the effects of a composition provided herein. In some embodiments, five or more markers are measured to assess the effects of a composition provided herein.

In some embodiments, histological analysis is performed to assess the effects of a composition provided herein. In some embodiments, blood sample analysis is performed to assess the effects of a composition provided herein. In some embodiments, an immune-response is performed to assess the effects of a composition provided herein.

Creatinine level: In some embodiments, effects of the treatments disclosed herein are ascertained by serum creatinine level in a patient receiving the treatment. Phosphocreatine, also known as creatine phosphate (CP) or PCr (Pcr), is a phosphorylated creatine molecule that serves as a rapidly mobilizable reserve of high-energy phosphates in skeletal muscle and the brain. Creatinine is a breakdown product of creatine phosphate in muscle, and is usually produced at a fairly constant rate by the body (depending on muscle mass). For example, a reduction of creatinine level indicates improvement in a subject with active lupus nephritis. In some embodiments, low dose of LAQ is used to maintain normal creatinine level.

Serum or plasma creatinine measurement: Serum creatinine (a blood measurement) can be easily-measured by-product of muscle metabolism. Creatinine itself is an important biomolecule because it is a major by-product of energy usage in muscle, via a biological system involving creatine, phosphocreatine (also known as creatine phosphate), and adenosine triphosphate (ATP, the body's immediate energy supply). Measuring serum creatinine is a simple test and it is the most commonly used indicator of renal function. A rise in blood creatinine level is observed only with marked damage to functioning nephrons. Therefore, this test is unsuitable for detecting early-stage kidney disease. A better estimation of kidney function is given by the creatinine clearance (CrCl) test. Creatinine clearance can be accurately calculated using serum creatinine concentration and some or all of the following variables: sex, age, weight and race, as suggested by the American Diabetes Association without a 24-hour urine collection. Some laboratories will calculate the CrCl if written on the pathology request form, and the necessary age, sex, and weight are included in the patient information.

A concern as of late 2010 relates to the adoption of a new analytical methodology, and a possible impact this may have in clinical medicine. All clinical laboratories in the US will soon use a new standardized isotope dilution mass spectrometry (IDMS) method to measure serum creatinine IDMS appears to give lower values than older methods when the serum creatinine values are relatively low, for example 0.7 mg/dl. The IDMS method would result in a comparative overestimation of the corresponding calculated glomerular filtration rate (GFR) in some patients with normal renal function. A few medicines are dosed even in normal renal function on that derived GFR. The dose, unless further modified, could now be higher than desired, potentially causing increased drug-related toxicity. To counter the effect of changing to IDMS, new FDA guidelines have suggested limiting doses to specified maxima with carboplatin, a chemotherapy drug.

Urine creatinine measurement: Creatinine concentration is also checked during standard urine drug tests. Normal creatinine levels indicate the test sample is undiluted, whereas low amounts of creatinine in the urine indicate either a manipulated test or low individual baseline creatinine levels. Test samples considered manipulated due to low creatinine are not tested, and the test is sometimes considered failed.

Diluted samples may not always be due to a conscious effort of subversion, [citation needed] and diluted samples cannot be proved to be intentional, but are only assumed to be. Random urine creatinine levels have no standard reference ranges. They are usually used with other tests to reference levels of other substances measured in the urine. Diuretics, such as coffee and tea, cause more frequent urination, thus potently decreasing creatinine levels. A decrease in muscle mass will also cause a lower reading of creatinine, as will pregnancy.

Additional details concerning Creatinine measurements can be found, for example, in Taylor, E. Howard (1989). Clinical Chemistry. New York: John Wiley and Sons. pp. 4, 58-62 and Gross J L, de Azevedo M J, Silveiro S P, Canani L H, Caramori M L, Zelmanovitz T (January 2005). “Diabetic nephropathy: diagnosis, prevention, and treatment,” Diabetes Care 28 (1): 164-176, each of which is hereby incorporated by reference in its entirety.

Anti-dsDNA antibodies: Anti-dsDNA antibodies are a group of anti-nuclear antibodies and their target antigen is double stranded DNA. The presence of dsDNA is highly diagnostic of systemic lupus erythematosus (SLE) and is implicated in the pathogenesis of lupus nephritis.

In some embodiments, absence or reduction of anti-dsDNA antibodies from a subject who has been previously diagnosed with such antibodies can be used as an indicator for monitoring the effectiveness of a treatment. For example, total absence indicates cure while drastic reduction suggests significant improvement of conditions.

Anti-dsDNA antibodies are highly associated with glomerulonephritis in SLE, although some patients with high titers of anti-dsDNA antibodies do not develop renal disease. This is most likely due to the fact that anti-dsDNA are a heterogeneous population, some of which have been found not to be pathogenic. Anti-dsDNA antibodies can be present in normal individuals, however these antibodies are usually low avidity IgM isotype. In contrast, pathogenic anti-dsDNA antibodies found in SLE are usually of IgG isotype and show high avidity for dsDNA. One possible mechanism for anti-dsDNA and their role in nephritis is the formation of immune complexes that arise by indirect binding to DNA or nucleosomes that are adhered to the glomerular basement membrane (GBM). Another mechanism is direct binding of antibodies to GBM antigens such as C1q, nucleosomal proteins, heparin sulphate or laminin, which can initiate an inflammatory response by activating complement. They can also be internalized by certain molecules on the GBM cells and cause inflammatory cascades, proliferation and alteration of cellular functions.

Blood tests such as enzyme-linked immunosorbent assay (ELISA) and immunofluorescence are routinely performed to detect anti-dsDNA antibodies in diagnostic laboratories.

A number of assays can be used to measure the quantity of anti-dsDNA antibodies in serum, including and not limited to the following:

Farr Assay: The Farr assay is used to quantify the amount of anti-dsDNA antibodies in serum. Ammonium sulphate is used to precipitate antigen-antibody complexes that form if the sera contains antibodies to dsDNA. The quantity of these antibodies is determined by using radioactively labeled dsDNA. Although this test is very specific, it is of little use in routine diagnostic laboratories due to its laboriousness and use of radioactive materials. The Farr assay is one of the only tests available that detects high avidity antibodies (along with Crithidia luciliae) and also has the ability to detect antibodies of any isotype.

PEG Assay: The polyethylene glycol (PEG) assay precipitates DNA-antibody complexes, similar to the Farr Assay. However, unlike the Farr Assay it does not dissociate the low avidity antibody complexes, allowing for the detection of both high and low avidity anti-dsDNA antibodies.[24]

Animal Tissue Assay: Animal tissue was the first substrate for immunofluorescent detection of antinuclear antibodies and has been in use since the late 1950s. Liver and kidney tissue sections from animals such as rats are used to identify anti-dsDNA antibodies. This substrate has largely been superseded by the use of HEp-2 cells.

HEp-2 Assay: Hep-2 cells, originally of laryngeal carcinoma origin, are actually a contamination of HeLa cells.[25] They are routinely used in the diagnosis of ANA in diagnostic laboratories. HEp-2 cells provide a greater ability to differentiate patterns of ANA than animal sections, due to the large nuclei and high mitotic rate of the cell line. Upon incubation with serum containing anti-dsDNA antibodies and fluorescent labeled secondary antibodies, homogeneous staining of interphase nuclei and condensed chromosomal staining of mitotic cells can be seen.

Crithidia Assay: Crithidia luciliae is a heamoflagellate protist with an organelle known as the kinetoplast. This organelle contains a high concentration of circular DNA with no recognizable nuclear antigens, allowing for the reliable detection of anti-dsDNA antibodies. The kinetoplast fluoresces if serum contains high avidity anti-dsDNA antibodies. This test has a higher specificity than EIA because it uses unprocessed DNA. Processed DNA can contain regions of ssDNA, allowing detection of anti-dsDNA antibodies, which can give false positive results.

EIA Assay: EIA detects antibodies using a DNA-coated polystyrene micro-titer plate. The DNA used in these assays is often recombinant dsDNA or from calf thymus extract. Upon incubation with serum containing anti-dsDNA antibodies, the antibodies will bind to the DNA and can then be visualized using enzyme-linked secondary antibodies. This assay can be quantitative or semi-quantitative, allowing for estimations of the levels of anti-dsDNA antibodies. This test can produce false positives due to contamination of ssDNA from denatured dsDNA. EIA detects low and high avidity anti-dsDNA antibodies, increasing its sensitivity and reducing its specificity.

Flow Cytometry Assay: Flow cytometry for the detection of ANA uses multiplexed polystyrene beads coated with multiple auto-antigens, such as SSA, SSB, Sm, RNP, Sc1-70, Jo-1, dsDNA, centromere B and histone. Serum is incubated with the beads and in the presence of anti-dsDNA antibodies, or any other ANA, the antibodies will bind and fluorescent labeled secondary antibodies will be used for detection. The beads are run through a flow cell which uses a laser to detect fluorescence.[29][30]

Multiplex Immunoassay (MIA): Similar to the flow cytometry method of ANA detection, the MIA uses wells containing auto-antigens and HEp-2 extract coated beads. The bead sets are coated with specific auto-antigens and can be detected individually to allow identification of the particular autoantibody. Automated analysis of the well fluorescence allows for rapid detection of autoantibodies.

Microarrays Assay: Microarrays are a newly emerging method for the detection of ANA. Individual auto-antigens are painted in an array of dots onto a surface such as polystyrene. A single array could consist of hundreds of auto-antigens for screening of multiple autoimmune diseases simultaneously. If anti-dsDNA antibodies are present, incubation of serum and the microarray allow for binding and the dots can then be visualized using a fluorescent labeled anti-IgG antibody.

Additional details can be found, for example, in Kavanaugh A, Tomar R, Reveille J, Solomon D H, Homburger H A (January 2000), “Guidelines for clinical use of the antinuclear antibody test and tests for specific autoantibodies to nuclear antigens. American College of Pathologists,” Arch. Pathol. Lab. Med. 124 (1): 71-81; Mortensen E S, Fenton K A, Rekvig O P (February 2008), “Lupus nephritis: the central role of nucleosomes revealed,” Am. J. Pathol. 172 (2): 275-283; Egner W (June 2000), “The use of laboratory tests in the diagnosis of SLE,” J. Clin. Pathol. 53 (6): 424-432; Slater N G, Cameron J S, Lessof M H (September 1976), “The Crithidia luciliae kinetoplast immunofluorescence test in systemic lupus erythematosus,” Clin. Exp. Immunol. 25 (3): 480-486; Burnett, David; Crocker, John R. (1999). The Science of Laboratory Diagnosis. ISIS Medical Media. pp. 494-495; Hueber W, Utz P J, Steinman L, Robinson W H (2002), “Autoantibody profiling for the study and treatment of autoimmune disease,” Arthritis Res. 4 (5): 290-295; each of which is hereby incorporated by reference in its entirety.

The invention is described in more detail in the following illustrative examples. Although the examples can represent only selected embodiments of the invention, it should be understood that the following examples are illustrative and not limiting.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Laquinimod in Preventing and Suppressing Murine Lupus Nephritis

The studies in this example described below show Laquinimod (LAQ) is equivalent to mycophenolate mofetil (MMF) in preventing and suppressing murine lupus nephritis and has greater effects on myeloid/monocyte/macrophage cells.

Lupus nephritis (LN) depends on autoAb deposition and activation of multiple cell types that infiltrate kidneys and promote inflammation—monocytes/macrophages (MM), DCs, T and B cells. Laquinimod (LAQ) administered to humans down-regulates Ag presentation, decreases chemokine production, decreases MHC expression on MM, and induces apoptotic pathways in PBMC (Gurevich M et al 2010). LAQ reduces progression of relapsing remitting multiple sclerosis (Comi G et al NEJM 2012); it is currently in clinical trials in SLE. MMF targets primarily lymphocytes; it is effective in many LN patients.

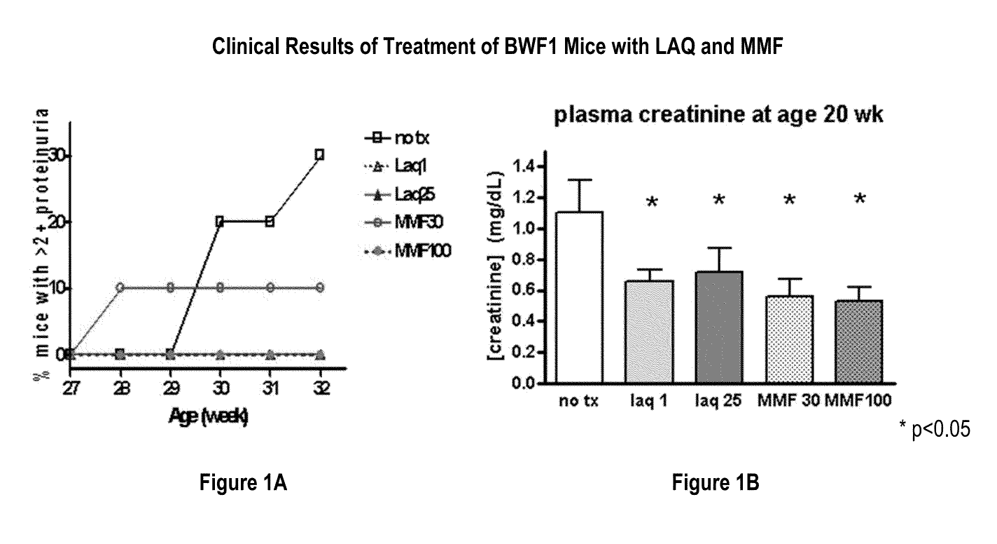

Clinical and immune cell changes are compared in groups of 10-12 BWF1 female mice treated orally 3 times a week for 24 weeks with a) water; b) LAQ 1 mg/kg; c) LAQ 25 mg/kg; d) MMF 30 mg/kg; e) MMF100 mg/kg. Survival was better in both LAQ groups and the MMF100 group vs. controls (p=0.028). LAQ at both doses was equivalent to MMF100 in preventing proteinuria in mice treated before disease appeared. At 32 wks. of age 50% of mice on water had proteinuria vs. zero in LAQ and MMF100 groups (p<0.0001) Renal histology mirrored proteinuria: mean total histologic scores were 7.8 on water, 1.0 on LAQ and 0.9 on MMF100 (p<0.01 both treatment groups compared to controls). Glomerular deposition of Ig and C3 were in the normal range in LAQ and MMF, but significantly increased in the water group (p<0.001). Both MMF and LAQ suppressed serum anti-DNA antibody production. See, e.g., FIG. 1A.

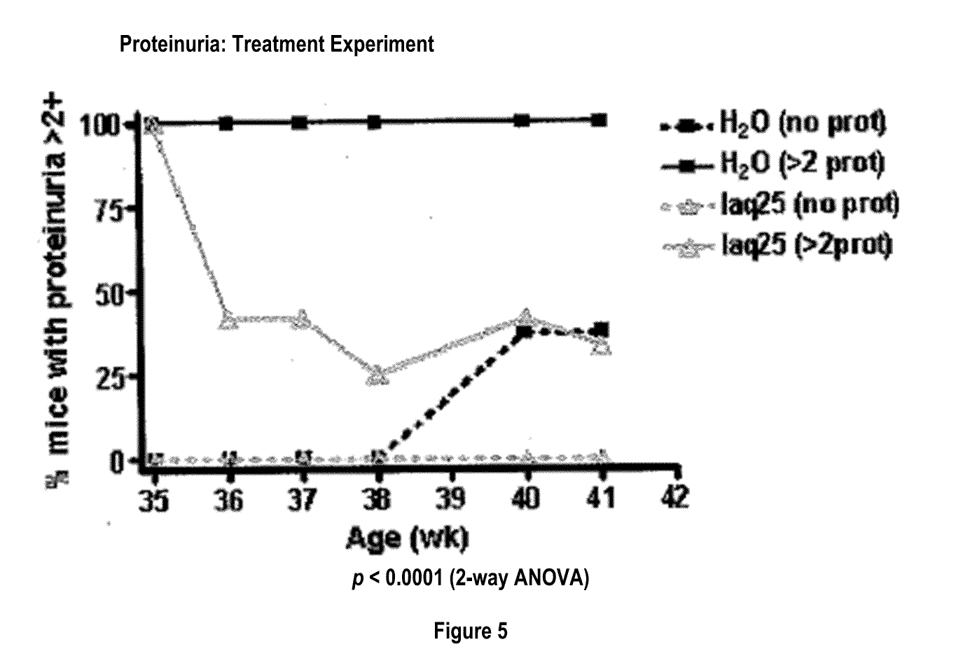

Mice treated after clinical nephritis appeared (≧3+ proteinuria) improved on LAQ: after 3 wks. of treatment proteinuria was present in 100% on water vs. 25% on LAQ (p<0.001). Survival was also better in mice treated with LAQ (p<0.0001). See, e.g., FIG. 5.

### Example 2

### Comparison Between LAQ and MMF

Effects on splenic PBMC differed between LAQ and MMF (Tables 1 and 2). Neither treatment changed total numbers of B cells. MMF decreased CD4+ and CD8+ T cell percentage; LAQ did not. LAQ compared to MMF increased numbers of two putative regulatory cells, CD4⁺CD25⁺Foxp3⁺ Treg and CD11b⁺Ly6C^{int}GR-1⁺ myeloid MM. Most interesting was the observation that LAQ, but not MMF, significantly reduced numbers of MM.

TABLE 1
Frequency of Spleen Cell Subsets.

| | Frequency of cells ± SEM | | |
|---|---|---|---|
| | Control | LAQ | MMI- |
| | untreated mice | treated mice | treated mice |
| Subsets | (n = 10) | (n = 9) | (n = 8) |

Myeloid cells

| | | | |
|---|---|---|---|
| CD11b⁺CD103⁻ (MΦ¹) | 3.86 ± 0.27 | 2.63 ± 0.30^{A} | 2.26 ± 0.25^{A} |
| CD11c⁺CD103⁺ (DC) | 1.57 ± 0.29 | 0 92 ± 0.14 | 0.48 ± 0.04^{A} |
| CD11b⁺Ly6C^{hi}Gr-1⁻ (MΦ²) | 13.74 ± 2.07 | 12.15 ± 2.11 | 11.81 ± 1.58 |
| CD11b⁺Ly6C^{int}Gr-1⁻ (MΦ³) | 28.61 ± 3.39 | 18.48 ± 2.31^{A} | 23.44 ± 1.89 |
| CD11b⁺Ly6C^{int}Gr-1⁺ (Gr-1) | 17.58 ± 2.65 | 41.50 ± 3.08^{A} | 25.01 ± 5.32 |

B-cell subpopulations

| | | | |
|---|---|---|---|
| CD 19⁺ | 65.54 ± 4.62 | 68.02 ± 3.83 | 57.38 ± 3.89 |
| CD19⁺CD23⁺CD21⁺IgM^{hi} (T2) | 32.62 ± 4.40 | 51.19 ± 4.32^{A} | 36.56 ± 4.92 |
| CD19⁺CD23⁻CD21⁺IgM^{hi} (MZ) | 6.49 ± 1.96 | 11.85 ± 2.15 | 3.78 ± 0.99 |

NZBWF1 mice were treated by oral gavage with LAQ at dose of 25 mg/kg (3 days per week) or with MMF at dose of 100 mg/kg (5 days/week) starting at age 11 weeks. Water-gavaged mice constituted the control untreated animals. The spleen cell subsets were identified by the expression of cell surface markers at age 32 weeks as follow: iTregs; nTregs; MΦ: CD11b⁻CD103⁻; MΦ²: CD11b⁺Ly6C^{hi}Gr-1⁻; MΦ³: CD11b⁺Ly6C^{int}Gr-1⁻; Gr-1: CD11b ly6C^{int}Gr-1⁺; DC: CD11c⁺CD103⁺; T2: CD19⁺CD23⁺CD21⁺IgM^{hi}; MZ: CD19⁺CD23⁻CD21⁺IgM^{hi}. Abbreviations: iTregs=induced T regulatory cells (Tregs). nTregs=natural Tregs. T2=transitional 2 B cells. MZ=marginal zone B cells. MΦ=macrophages. Gr1=granulocytes. Values are mean±SEM. The statistical significance between the groups was determined by analysis of variance (-A-NOVA). ^{A}P<0.05 versus untreated controls.

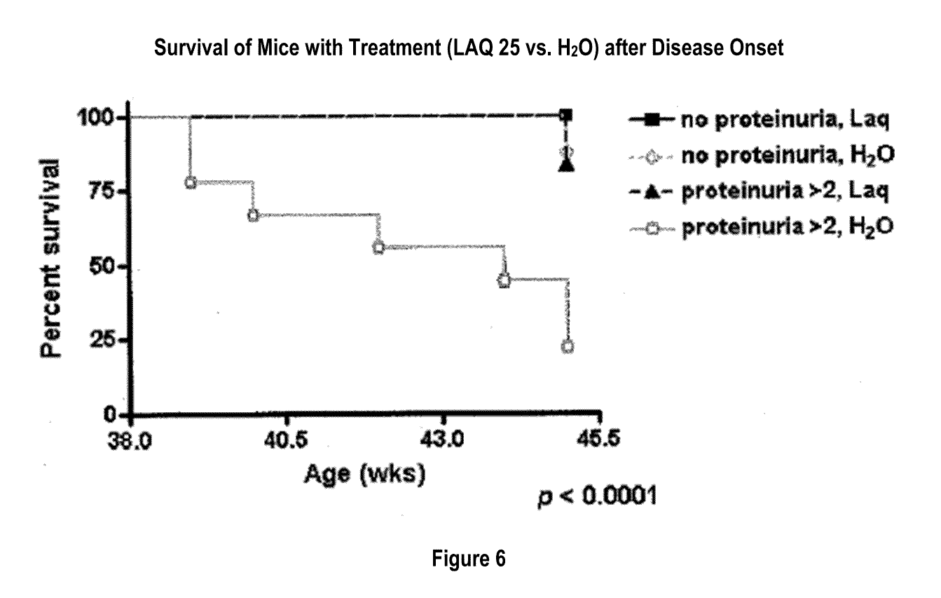

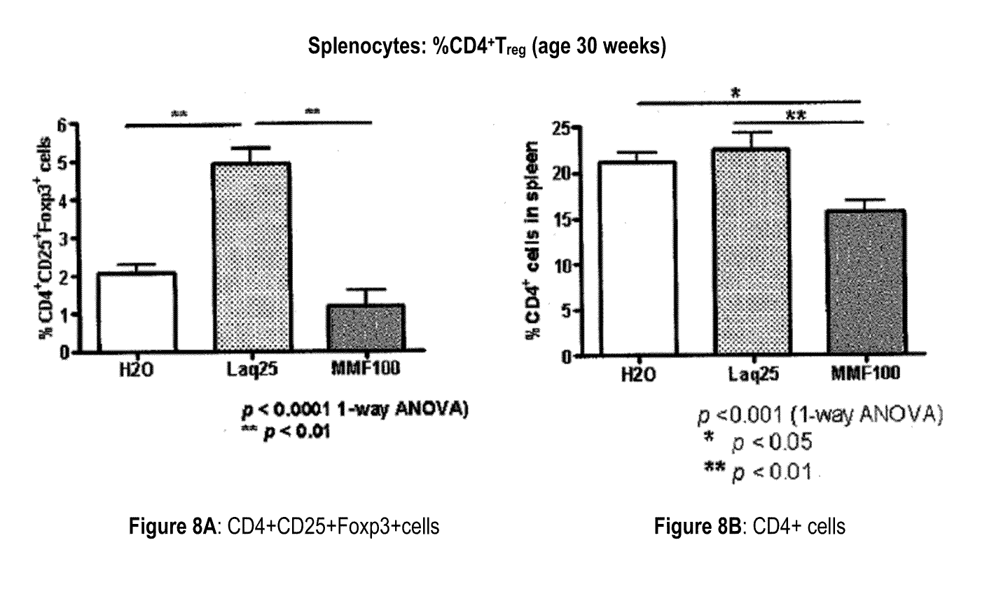

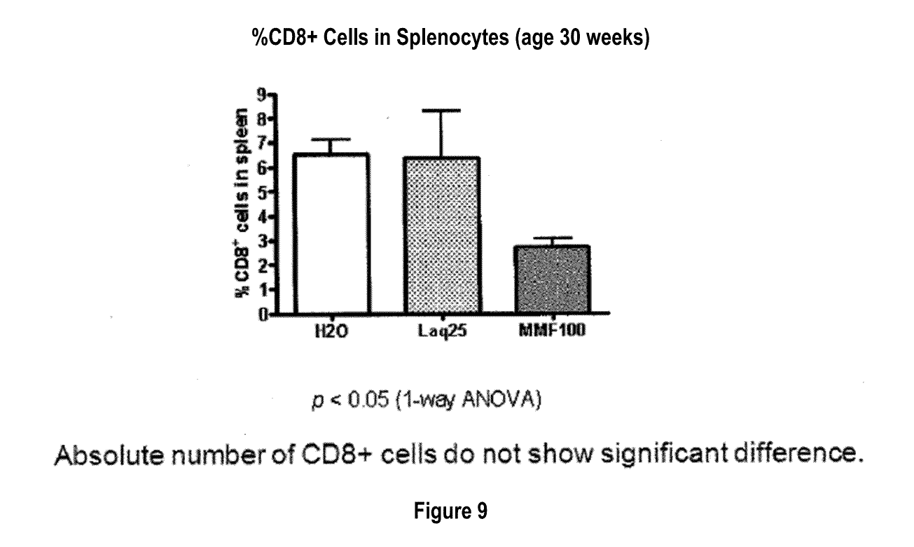

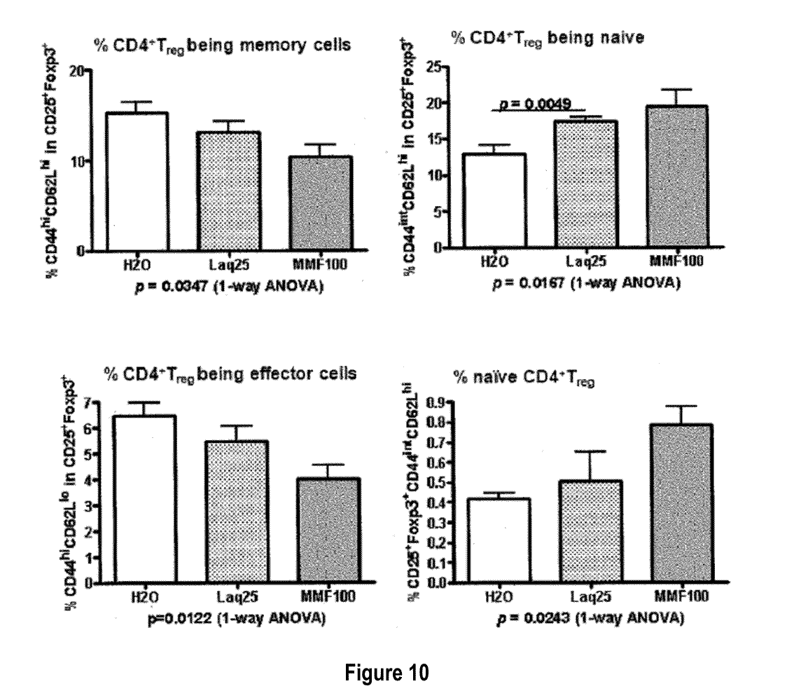

In summary, LAQ was highly effective in preventing and suppressing proteinuria and glomerular immune disease in BWF1 mice. Responses to MMF in high dose were similarly good. However, LAQ reduced numbers of MM, and MMF did not. In addition, LAQ induced different types of regulatory cells, distinguishing it from MMF. Since suppression of MM is likely to reduce renal inflammation and damage, future development of LAQ as a therapeutic for lupus nephritis is especially promising. These results are summarized in FIGS. 1-12.

### Example 3

### Assessment of Effect of Laquinimod on SLE in Animal Models

Systemic Lupus Erythematosus (SLE) is a disorder of generalized autoimmunity characterized by defective T cell-mediated responses and the formation of a variety of antibodies reactive to self or altered self-antigens. SLE is mainly characterized by the presence of anti-DNA antibodies. Some of these auto-antibodies combine with the corresponding auto-antigens, forming immune complexes, either in the circulating blood or directly in tissues, resulting in severe damage. Glomerulonephritis induced by immune complexes is in fact the major cause of death in patients with SLE. (NZB×NZW)FI (BWF1) mice are lupus-prone mice; females develop an SLE-like disease spontaneously including anti-dsDNA antibodies (Abs), proteinuria and Immune Complex Deposits (ICD). The (NZB×NZW)FI (NZB/W) murine model is the hallmark of spontaneous polygenic SLE.

Lupus nephritis (LN) depends on autoantibody (autoAb) deposition and activation of multiple cell types that infiltrate kidneys and promote inflammation—monocytes/macrophages (MM), Dendritic cells (DCs), T and B cells. The antibodies also activate intrinsic renal cells, including endothelial, mesangial, and renal tubular epithelial cells.

Laquinimod is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851.

Laquinimod administered to humans down-regulates antigen (Ag) presentation, decreases chemokine production, decreases Major histocompatibility complex (MHC) expression on MM, and induces apoptotic pathways in peripheral blood mononuclear cells (PBMC) (Gurevich M et al 2010). Laquinimod also reduces progression of relapsing remitting multiple sclerosis (Comi G et al NEJM 2012).

Mycophenolate Mofetil (MMF) targets primarily lymphocytes and is effective in many LN patients.

### Procedures

In this study, the effect of two closes of laquinimod in the (NZB×NZW)F1 model for SLE were assessed. The study also included a negative control (water) and a positive control (Mycophenolate Mofetil or MMF).

Clinical and immune cell changes were compared in groups of 10-12 BWF1 female mice in the following groups:
Group 1: Water orally 3 times a week for 24 weeks.
Group 2: Laquinimod 1 mg/kg orally 3 times a week for 24 weeks.
Group 3: Laquinimod 25 mg/kg orally 3 times a week for 24 weeks.
Group 4: Mycophenolate Mofetil 30 mg/kg orally 3 times a week for 24 weeks.
Group 5: Mycophenolate Mofetil 100 mg/kg orally 3 times a week for 24 weeks (“MMF 100”).

### Results

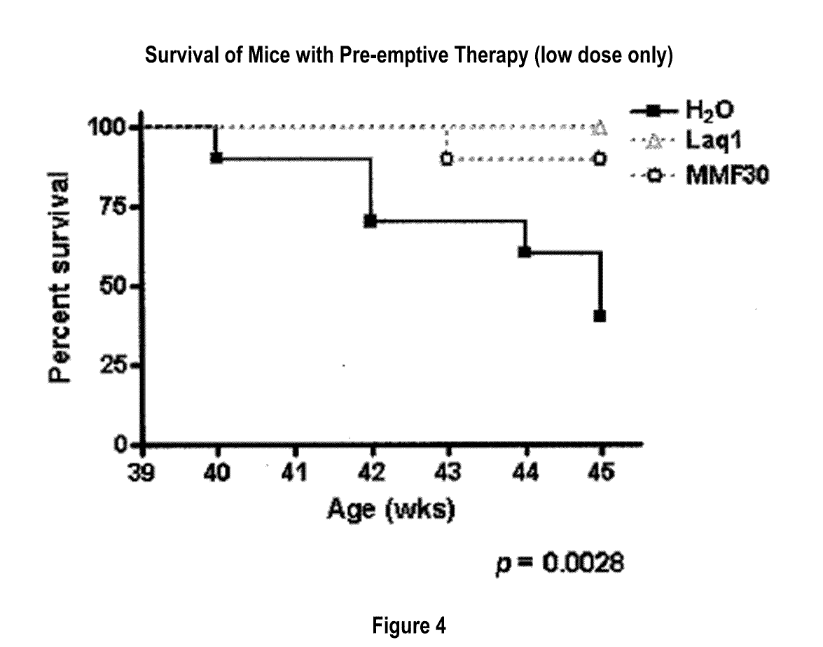

1. Survival was better in both laquinimod groups and the MMF 100 group as compared to the control group (p=0.028). See, e.g., FIG. 4.

2. Laquinimod at both doses was equivalent to MMF 100 in preventing proteinuria in mice treated before disease appeared.

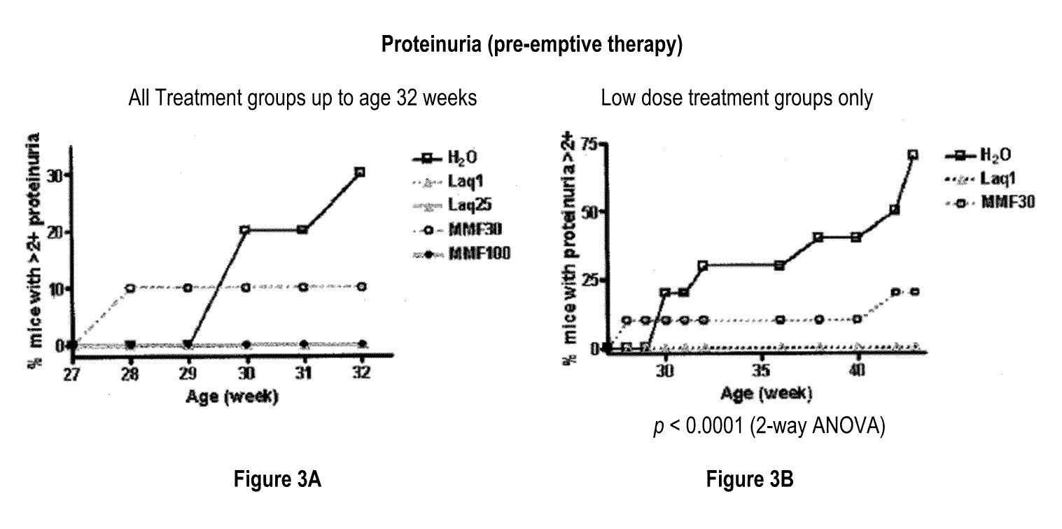

3. At 32 weeks of age 50% of mice in Group 1 had proteinuria while none had proteinuria in Groups 2, 3 and 5 (p<0.0001). See, e.g., FIG. 3A.

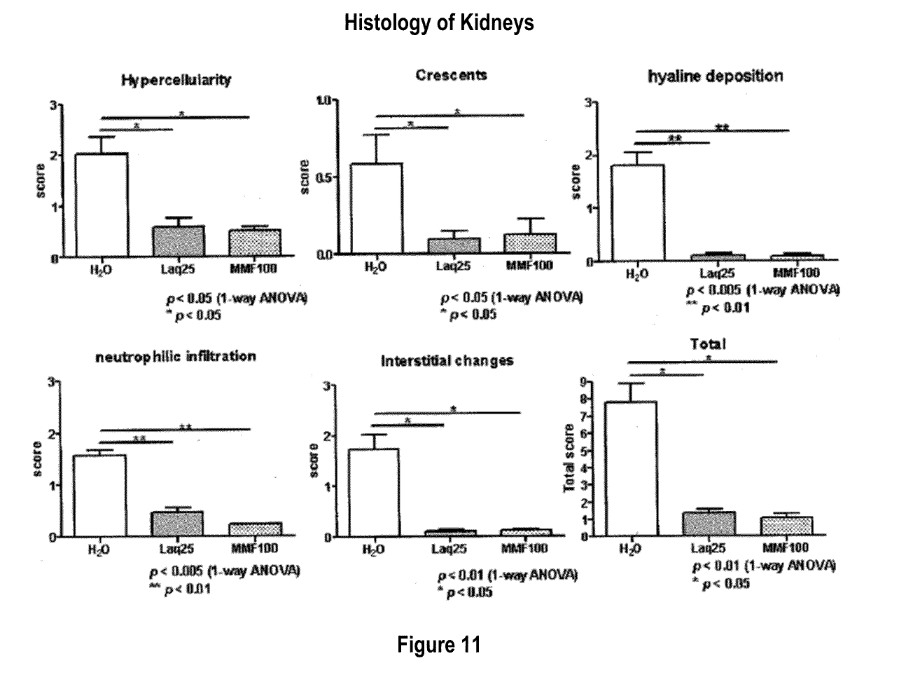

4. Renal histology mirrored proteinuria: mean total histologic scores were 7.8 for Group 1, 1.0 for laquinimod and 0.9 for Group 5 (p<0.01 both treatment groups compared to controls). See, e.g., FIG. 11.

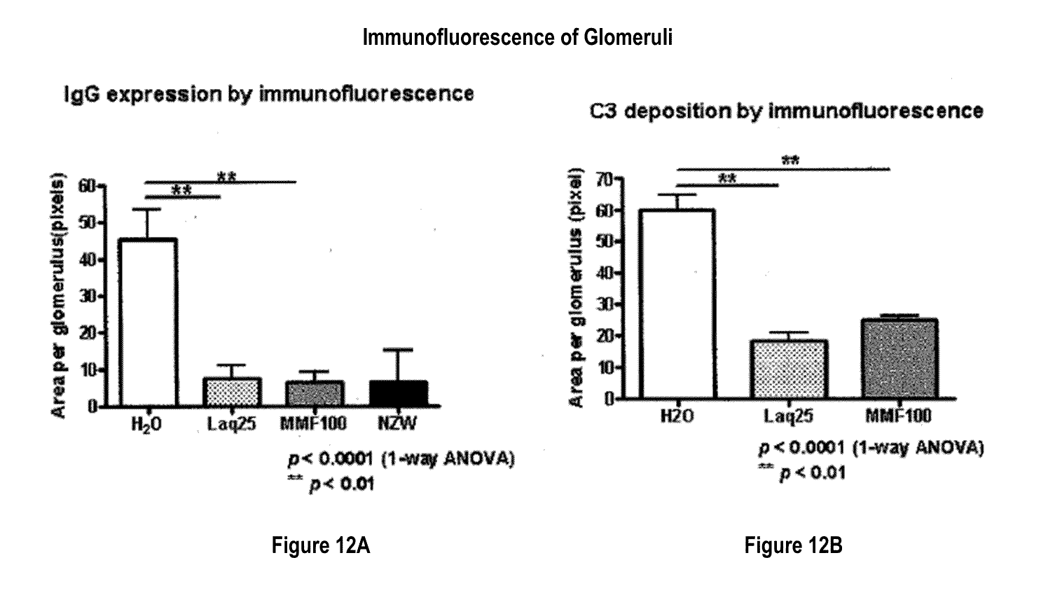

5. Glomerular deposition of immunoglobulin (Ig) and Complement component 3 (C3) were in the normal range in laquinimod and MMF groups, but is significantly increased in the water group (p<0.001). See, e.g., FIGS. 12A and 12B.

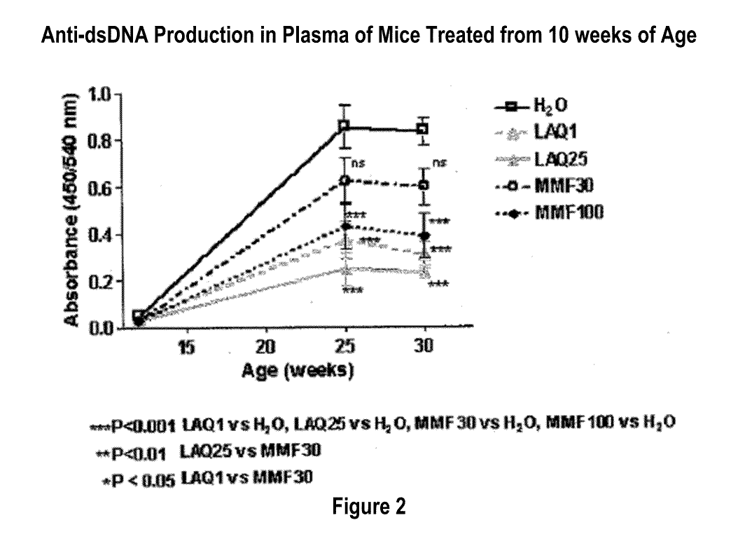

6. Laquinimod suppressed serum anti-dsDNA antibody production 5 better than MMF. See, e.g., FIG. 2.

7. Mice treated after clinical nephritis appeared (≧3+ proteinuria) improved with laquinimod treatment: after 3 weeks of treatment proteinuria was present in 100% of mice in Group 1 versus only 25% for laquinimod (p<0.001). Survival was also better in mice treated with laquinimod (p<0.0001). See, e.g., FIG. 5.

8. Effects on splenic PBMC differed between laquinimod and MMF. Neither treatment changed the total number of B cells. MMF decreased the percent of CD4+ T cells and CD8+ T cells, while laquinimod did not.

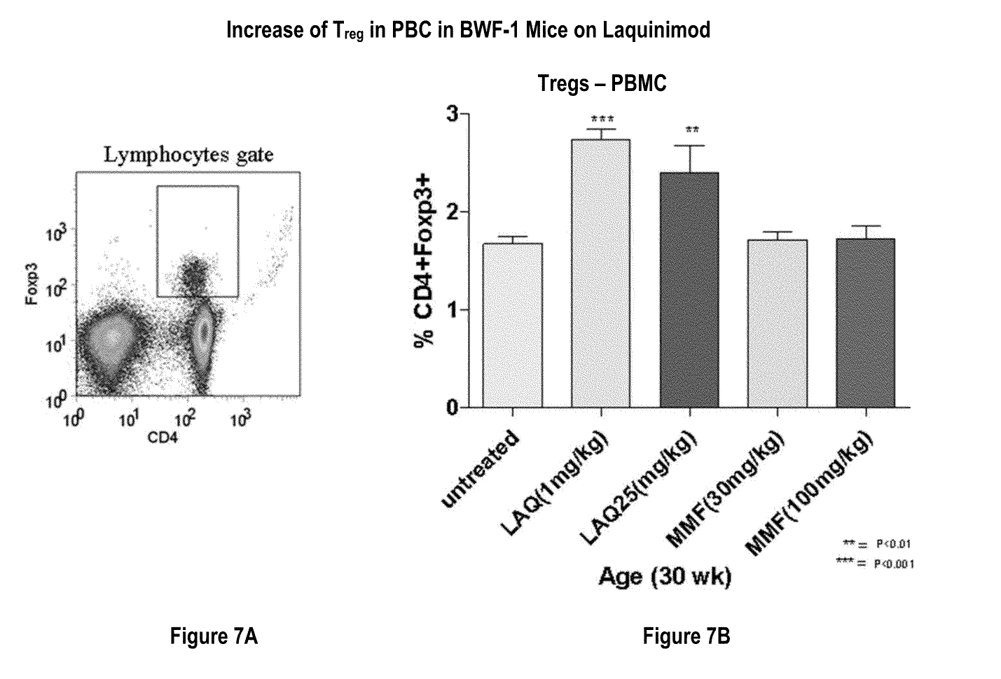

9. Laquinimod as compared to MMF increased the number of two putative regulatory cells, CD4⁺CD25⁺Foxp3⁺ Treg and CD11b⁺Ly6^{int}GR-1⁺ myeloid MM. An interesting observation was that laquinimod, but not MMF, significantly reduced the number of MM in the kidneys, as well as promoting change from M1 to M2 phenotypes in MM isolated from kidneys. See, e.g., FIGS. 7-10.

### Summary/Discussion

In summary, laquinimod was highly effective in preventing and suppressing proteinuria and glomerular immune disease in BWF1 mice. Response to MMF at the high dose was similarly good. However, laquinimod reduced the number of MM while MMF did not. In addition, laquinimod induced different types of regulatory cells, distinguishing it from MMF. Also, MMF is associated with gastrointestinal side effects, which are dose-dependent, occurring in up to 20% of patients at the dose of 2 g daily (Orvis, 2008). In contrast, laquinimod has better safety and tolerability profile and is associated with fewer side effects. Since suppression of MM is likely to reduce renal inflammation and damage, laquinimod is a promising prophylactic and therapeutic agent, for active lupus nephritis.

This experiment provides an important and surprising finding that laquinimod can delay or prevent SLE from progressing to active lupus nephritis, which is one of the most serious complications caused by SLE. Prior to this invention there is no indication in literature that laquinimod is capable of delaying or preventing active lupus nephritis.

### Example 4

### Laquinimod Induces Myeloid-Derived Suppressor Cells in the Kidney and Delays Nephritis in Lupus-Prone Mice

Objective: Lupus nephritis depends on autoantibody deposition and activation of multiple immune cell types that promote kidney inflammation, including monocytes/macrophages (M/M). Laquinimod (LAQ), currently in clinical trials for multiple sclerosis, blocks inflammatory M/M from entering the spinal cord. Activated M/M often infiltrate the kidney during SLE nephritis, and eliminating M/M-driven tissue damage in SLE nephritis could have greater therapeutic benefit versus currently utilized SLE treatments such as the lymphocyte-specific compound mycophenolate mofetil (MMF).

Methods: The murine SLE model BWF1, in which disease manifests as nephritis, was used to test LAQ efficacy. Preventive and therapeutic studies were performed to examine if LAQ could prevent or delay nephritis, measured by proteinuria, serum creatinine, survival, and renal pathology. Flow cytometry was utilized to identify leukocyte populations in kidney, and T cell suppression assays were performed with myeloid-derived suppressor cells (MDSC) isolated from spleens of treated mice.

Results: LAQ prevented or delayed lupus manifestations as well as or better than MMF. LAQ alone reduced M/M numbers in kidney and spleen while concurrently increasing MDSC numbers in both organs. Cytokine release after TLR stimulation in splenic M/M led to a shift from a type I M/M pro-inflammatory to a type II anti-inflammatory phenotype.

Conclusion: LAQ was effective in preventing and suppressing proteinuria and glomerulonephritis in BWF1 mice. LAQ reduced numbers of M/M and induced MDSC, distinguishing it from MMF. Future development of LAQ as a therapeutic for lupus nephritis is promising due to its unique suppression of inflammatory M/M that likely reduces renal damage.

Although glomerular inflammation in murine and human lupus nephritis is initiated by deposition of complement fixing immunoglobulin (Ig), including IgG anti-DNA, both inflammation and damage are perpetuated by activation of multiple additional pathways (1, 2). Glucocorticoid therapy suppresses many of these pathways and is broadly anti-inflammatory and immunosuppressive. However, it is not curative and is associated with many undesirable side effects. Therefore, it is now standard in humans to treat lupus nephritis with additional agents, including anti-malarials (which suppress antigen presentation and toll-like-receptor activation of dendritic cells) plus immunosuppressives (3). Currently standard of care often includes mycophenolate mofetil (MMF) or cyclophosphamide which primarily target lymphocytes. Since none of these treatments are curative, and many patients do not respond fully or sustain improvement, there is an unmet need for newer approaches.

Renal tissue-fixed macrophages are activated early in the course of lupus nephritis and contribute to tissue damage, as do infiltrating monocytes/macrophages (M/M), lymphocytes and neutrophils (4). Expansion of the resident macrophage population occurs in many forms of glomerulonephritis, including human lupus nephritis and is associated with renal injury (5). In addition, kidney disease is attenuated if M/M are not recruited, such as in MRL/lpr SLE-prone mice that are deficient in macrophage inhibitory factor (6). Therefore, an intervention that targets M/M plus lymphocytes might be advantageous. Laquinimod (5-chloro-N-ethyl-4-hydroxy-1-methyl-2-oxo-N-phenyl-1,2-dihydroquinoline-3-carboxamide) is an immunomodulatory drug which altered both lymphocytes and M/M in murine experimental autoimmune encephalomyelitis (EAE) (7, 8). It has been used successfully in a clinical trial in patients with multiple sclerosis with a mild adverse events profile (9). Therefore, laquinimod treatment can be equivalent to or better than MMF in treating lupus nephritis.

In the work reported here, NZB/NZW first generation females (BWF1), which develop lupus nephritis, was treated orally with water, MMF or laquinimod. Some mice were treated before clinical evidence of disease; others were treated after they were IgG anti-DNA positive or after they had heavy proteinuria.

Laquinimod was as effective as MMF, either in preventing onset of disease or in reducing renal disease in animals with advanced nephritis. Furthermore, while both MMF and laquinimod decreased lymphocyte numbers in the kidneys, only laquinimod reduced numbers of renal M/M. In addition, M/M populations were altered by laquinimod, with a lower proportion of pro-inflammatory M-1 phenotype cells and significantly higher proportions of myeloid—derived suppressor cells. Furthermore, M/M from mice treated with laquinimod were unable to respond to stimulation via TLR7 and -9 with production of TNFα, suggesting that laquinimod may prevent activation and migration of inflammatory M/M and delay nephritis (4). These results suggest that laquinimod has the potential to specifically target M/M and MDSC to delay or prevent human lupus nephritis.

### Mice

NZB (H-2^{d/d}), NZW (H-2^{z/z}) and NZB/W F₁ (H-2^{d/z}) (BWF₁) mice were purchased from the Jackson Laboratories (Bar Harbor, Me.). Mice were treated in accordance with the guidelines of the UCLA Animal Research Committee, an institution accredited by the Association for Assessment and Accreditation of Laboratory Animal Care (AAALAC). All experiments were conducted in female mice.

### Medication Treatment

Preventive Group: 11-week old (pre-nephritic) BWF₁ female mice were treated orally 3 times a week with a) water; b) laquinimod at 1 mg/kg (LAQ1, low dose) or c) 25 mg/kg (LAQ25, high dose) (Teva Pharmaceutical); or 5 times a week with d) mycophenolate mofetil at 30 mg/kg (MMF30, low dose) or e) 100 mg/kg (MMF100, high dose).

Therapeutic Groups: a low proteinuria group)(PU^{lo}) consisted of mice with IgG anti-dsDNA detectable in serum and proteinuria ≦100 mg/dL, and a high proteinuria group (PU^{hi}) had proteinuria ≧300 mg/dL. Mice in both therapeutic groups were treated with a) water, b) LAQ at 25 mg/kg 3 times a week, or d) MMF 100 mg/kg 5 times a week. Results from the prevention group showed these doses of LAQ and MMF were more effective than the lower doses.

### Reagents

The fluorescent-conjugated antibodies to CD11b, Ly6G, Ly6C, Gr1 (RB6-8C5), CD11c, CD3, CD4, CD8, CD19, Foxp3, IFN-γ, Arginase-1, IL-10, IL-12/23, TGFβ, TNFα used in flow cytometry experiments were from eBioscience, Biolegend or BD Biosciences.

Cell cultures were performed in RPMI 1640 (Cellgro) supplemented with L-glutamine (2 mM), penicillin (100 U/ml), streptomycin (0.1 mg/ml), 2-mercaptoethanol (Gibco) and 2% or 10% (v/v) fetal bovine serum.

### Cell Isolation, Proliferation and Cytokines

Single cell suspensions of splenocytes or kidney cells were prepared using cell strainers (Fisher), followed by red blood cell lysis. Kidney lymphocytes were separated from connective tissue by centrifugation (15 minutes at 400g) in 30% Percoll (GE Healthcare). CD4⁺CD25⁻ cells from spleen and kidney were isolated using the CD4⁺CD25⁺ regulatory T cell isolation kit according to manufacturer's protocol (Miltenyi). CD11b+ cells were isolated using EasySep mouse CD11b positive selection kit (StemCell Technologies), surface stained for Ly6C and Ly6G prior to sorting myeloid-derived suppressor cells (MDSC) using FACSAria II. Populations were >95% pure by FACS analysis.

CD4⁺CD25⁻ cells were labelled with 5 □M CFSE and stimulated with Dynabeads mouse T-cell activator CD3/CD28 (Invitrogen). Stimulated CD4⁺CD25⁻ cells (4×10⁴) were co-cultured with monocytic-MDSC(CD11b⁺Ly6C⁺Ly6G⁻) or granulocytic-MDSC(CD11b⁺Ly6C⁺Ly6G⁺) at a 1:1 ratio. Proliferation of CD4⁺ cells was assayed after 4 days of incubation at 37° C., 5% CO₂ by flow cytometry.

For intracellular cytokines, whole splenocytes were stimulated with a) LPS (1 μg/mL) (Sigma), imiquimod, a toll like receptor 7 (TLR7) agonist, or ODN, a TLR9 agonist (both at 10 μg/mL) in 2% FBS RPMI 1640 for 24 hours in the presence of monensin (BioLegend) for the last 12 hours; or b) phorbol 12-myristate 13-acetate (PMA, 50 ng/ml) plus ionomycin (500 ng/ml) in the presence of monensin for 5 hours. Monensin was used to prevent cytokine secretion and optimize intracellular staining Intracellular expression of cytokines in CD4⁺ cells and macrophages (CD11b⁺Ly6C⁻Ly6G⁻) was analyzed by intracellular flow cytometry.

### Proteinuria, Creatinine, Anti-dsDNA Antibodies and BUN

Proteinuria was evaluated weekly using Albustix (Siemens), serum creatinine using a commercial kit (Arbor Assays) and anti-dsDNA IgG antibodies by ELISA as described previously (10). See, e.g., FIGS. 1 and 2.

### Flow Cytometry

Single-cell suspensions from spleen or kidneys were stained for extracellular markers for 20 min at 4° C. in PBS/1% FBS. Intracellular staining for Foxp3 and cytokines was performed using the Foxp3 Fixation/Permeabilization kit according to the manufacturer's protocol (eBioscience). Cells were acquired on a FACS LSR II (BD) and data was analyzed using Flowjo (Tree Star).

### Immunoflourescence and Histology

Kidney specimens of BWF₁ mice (37 weeks of age in the prevention group or 7 and 11 weeks after institution of early or late therapies) were embedded in OCT compound and snap frozen. 4-5 μm an sections were air-dried, fixed with cold acetone and incubated with blocking buffer (anti-CD16/CD32 (1/50), 2% normal goat or rat serum, 2% BSA).

Immunoglobulin G and C3 complement glomerular deposition were investigated by incubation of cryosections with fluoroscein isothiocyanate (FITC)-conjugated goat anti-mouse IgG (1:50) (Sigma) or rat anti-mouse C3 (1:800) (Solulink) in blocking buffer for 2 hours at room temperature. Images were acquired using Nikon Eclipse TE2000-U microscope and MetaMorph 6.3 software (Sunnyvale, Calif.). Quantification of the area immunostained in tissue sections was made using the computer image analysis software ImageJ (rsb<dot>info<dot>nih<dot>gov</>ij</>index<dot>html). For histology, kidney sections were fixed in 10% formalin and 2 μm-paraffin sections were stained with H&E, PAS and methenamine silver (Jones) for histological analysis.

Histological analysis of glomerular pathology was done by an investigator blinded to treatment group and included acute inflammatory features (endocapillary hypercellularity, mesangial hypercellularity, cellular necrosis, cellular crescents, and interstitial inflammation), and chronic features (global glomerulosclerosis (GS), focal segmental glomerulosclerosis (FSGS), and interstitial fibrosis or tubular atrophy (IF/TA)). The severity of each feature was graded as 0 (absent), 1 (lesions involving up to 25% of the component evaluated), 2 (lesions in 26-50% evaluated) or 3 (lesions in >50%) in each field of the microscope at 400× magnification. The weighted total score of the clinical features was based on this formula: [endocapillary hypercellularity+mesengial hypercellularity+(cellular necrosis*2)+(cellular crescents*2)+interstitial inflammation)/5]+(GS+FSGS+IFTA)/3, with maximum score 5.2. A minimum of 25 glomeruli, of at least 10 mice per group, were scored. Scores from each individual mouse were added and averaged to yield the glomerulonephritis score.

### Statistical Analyses

Statistical analyses were performed using Prism 4 software (GraphPad). Comparisons between two groups were performed by the two-tailed t-test. Tests between more than two groups were performed by one-way analysis of variance (ANOVA). In cases where repeated measures were assessed, two-way ANOVA were used. Tukey's multiple comparison test (one-way ANOVA) and Bonferroni posttest (two-way ANOVA) were used in post-hoc testing between pairs of groups. Survival was analyzed by logrank test, and statistical significance was adjusted to account for multiple comparisons using the Bonferroni method. p values <0.05 were considered significant. With a sample size of 6 mice in each group, the minimally detectable (with 80% power) effect size is 1.33 assuming a two group t-test with a 0.05 two-sided significance level. The magnitude of the minimally detectable effect was smaller than that observed in pilot studies, suggesting that this sample size was sufficient.

### Results

### LAQ Suppresses Murine Lupus Nephritis in a Preventive Study

There were three treatment arms of the study: 1) the preventive study examined whether LAQ delayed onset of disease by treating young, pre-diseased mice; 2) the therapeutic low proteinuria)(PU^{lo}) arm tested whether treatment of mice with IgG anti-dsDNA but proteinuria ≦100 mg/dl could delay kidney damage; and 3) the therapeutic high proteinuria (PU^{hi}) study examined if treatment could reverse disease in mice with anti-dsDNA and heavy proteinuria (≦300 mg/dL).

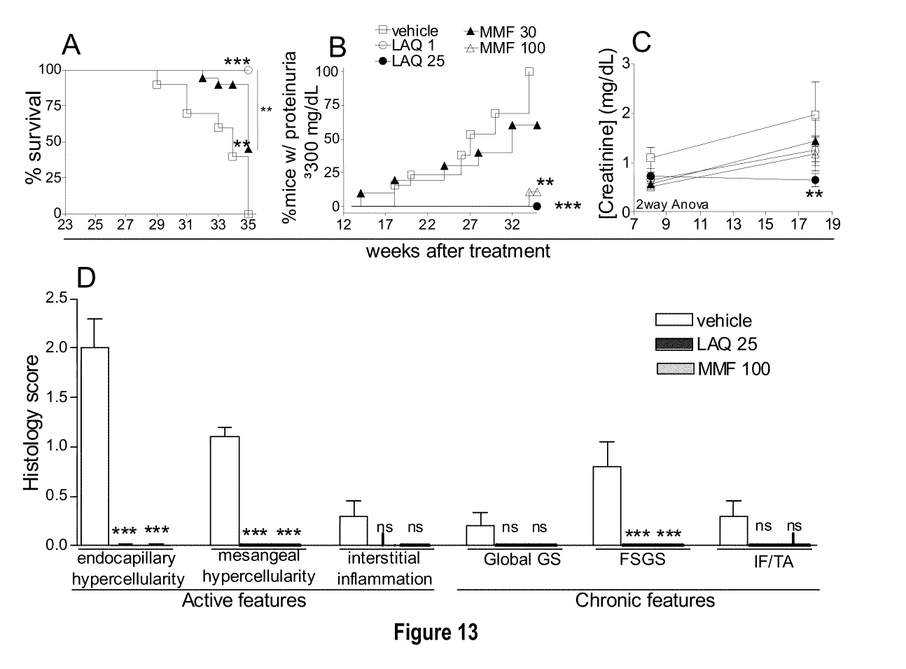

In the preventive study, anti-dsDNA serum levels were significantly lower in LAQ-treated mice versus vehicle-treated animals after 13-18 weeks of treatment and were similar to levels in mice treated with 100 mg/kg MMF (data not shown). Survival was significantly better in mice administered the low doses of LAQ and MMF versus water-fed mice (FIG. 13A). LAQ treatment (both doses) completely prevented proteinuria over the 18-week study period (FIG. 13B). MMF100 prevented proteinuria in a similar manner to both laquinimod doses, but MMF 30 did not (FIG. 13B). Rise in serum creatinine levels was significantly delayed only in LAQ25 treatment after 18 weeks of treatment (FIG. 13C).

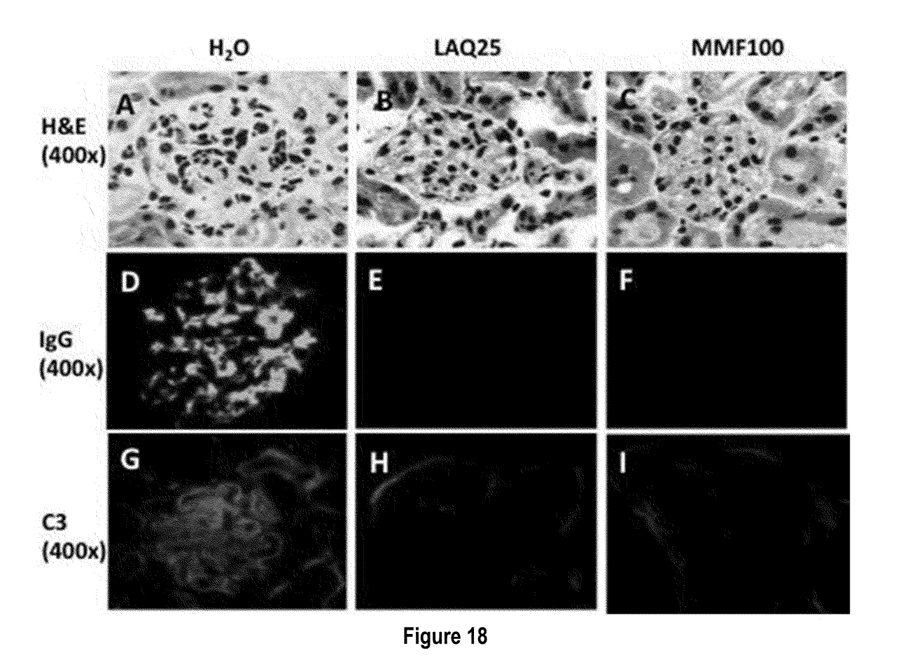

BWF1 mice develop renal failure with IgG and C3 deposition as well as acute and chronic pathological changes (11). Renal specimens from mice were obtained after more than 50% of the controls developed high proteinuria to examine kidney histology. Although kidney specimens from mice treated with LAQ25 or MMF100 were largely unremarkable, control mice had increased glomerular deposits of IgG and complement C3 (FIG. 18). In addition, water-treated controls developed acute pathological changes such as endo-capillary and mesangial hypercellularity and interstitial inflammation, and chronic damages such as focal segmental glomerulosclerosis and interstitial fibrosis or tubular atrophy (FIG. 13D). These data suggest that treatment with either laquinimod or MMF delays the development of lupus nephritis. Laquinimod appears to be at least as effective as MMF in delaying kidney damage in murine SLE. In summary, the preventive experiments illustrated that laquinimod treatment at both doses was as effective as MMF100 with regard to anti-dsDNA, survival, proteinuria, and kidney damage, but only LAQ25 prevented a rise in serum creatinine Thus, high doses of laquinimod (LAQ25) and MMF (MMF100) are used for the therapeutic arms of the study.

Therapeutic Treatment with LAQ in BWF1 Mice Delayed Disease Progression

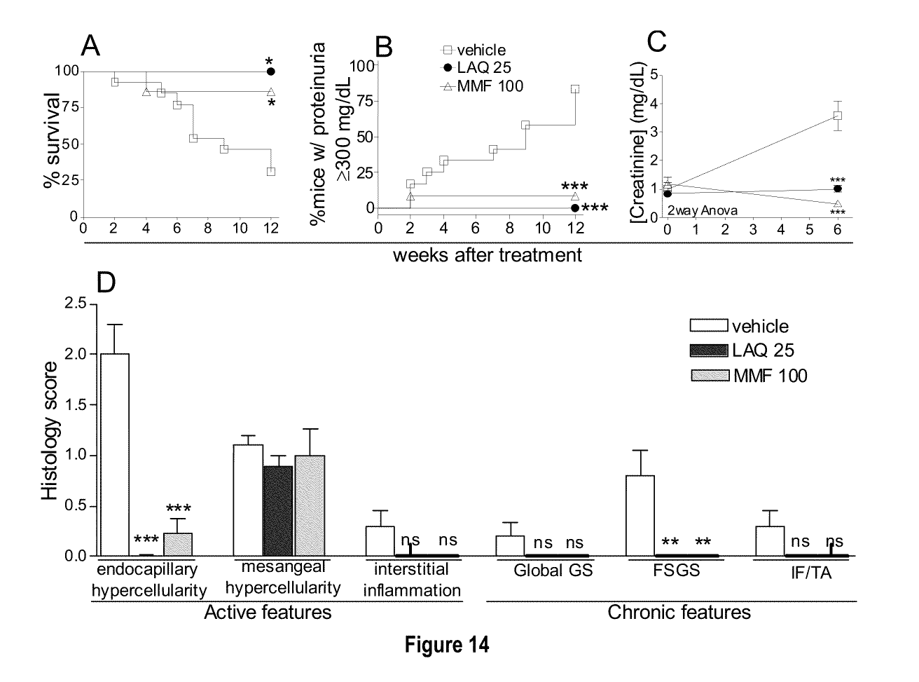

In the PU^{lo} arm, anti-dsDNA was not significantly decreased in LAQ25- or MMF100-treated mice (data not shown). However, laquinimod and MMF significantly prolonged survival (FIG. 14A), and prevented increased proteinuria (FIG. 14B) and rise in serum creatinine (FIG. 14C). LAQ25 and MMF100 were equally effective in reducing endo-capillary hypercellularity, interstitial inflammation, and focal segmental glomerulosclerosis in PU^{lo} mice (FIG. 14D). Necrosis and crescent formation were uncommon in all groups (data not shown).

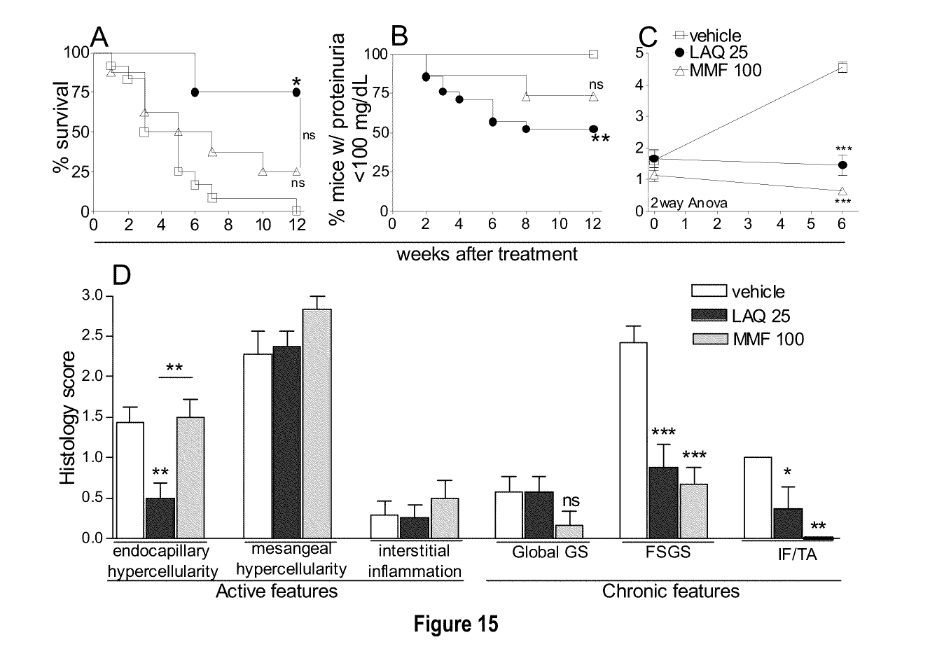

In the PU^{hi} arm, LAQ25 treatment significantly increased survival after 12 weeks of treatment, which was not observed in mice receiving MMF100 (FIG. 15A). Similarly, laquinimod treatment significantly reduced proteinuria to levels below 300 mg/dL in 50% of animals treated after 8 weeks of treatment compared to water-treated animals (FIG. 15B). Although approximately 25% of MMF-treated animals also experienced a drop in proteinuria below 300 mg/dL, this was not significant versus controls (FIG. 15B). Both treatments prevented a rise in serum creatinine (FIG. 15C). Endo-capillary hypercellularity in the kidney was significantly lower in laquinimod-treated mice versus both control and MMF-treated mice, and FSGS and IF/TA were significantly lower in mice treated with laquinimod and MMF compared to controls (FIG. 15D). Similar to the PU^{lo} arm, necrosis and crescent formation were uncommon events in all treatment groups. Together, these data show that laquinimod suppresses clinical and histologic manifestations of murine lupus nephritis and increases survival in therapeutic treatment regimens at levels equal to or better than MMF.

LAQ Induces Myeloid Cells Co-Expressing CD11b, Ly6C and/or Ly6G and Decreases Total Macrophages in Spleen and Kidney

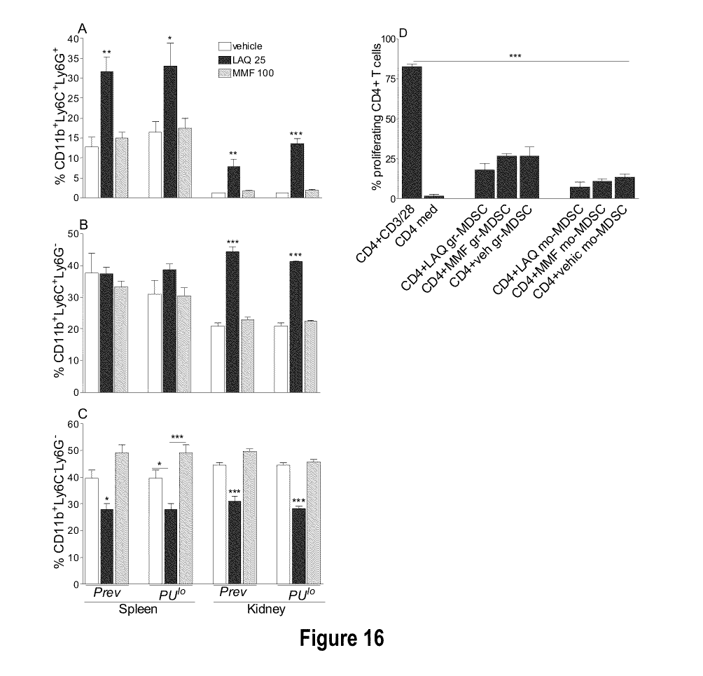

Previous studies have shown that laquinimod is an immunodulator that affects various leukocyte populations but preferentially targets antigen presenting cells in EAE models (7, 8, 12). The effect of laquinimod on different leukocyte subsets in kidney and spleen of BWF1 mice were investigated in the preventive and PU^{lo} groups after 10 and 25 weeks of treatment, respectively (control animals in the PU^{hi} arm died very rapidly after treatment with water was initiated, and these animals were not analyzed). In both spleen and kidney, there was a significant increase in the frequency of CD11b⁺Ly6C⁺Ly6G⁺ cells in mice treated with laquinimod in both PU^{lo} and PU^{hi} arms versus MMF- and vehicle-treated animals (FIG. 16A, Table 2, Table 3). CD11b⁺Ly6C⁺Ly6G⁺ cells were increased in kidneys of laquinimod-treated mice (FIG. 16B, Table 2). In contrast, a significant decrease in CD11b⁺Ly6C⁻Ly6G⁻ (CD11b⁺MDSC⁻) monocyte/macrophages was observed in both spleen and kidney of laquinimod-treated mice in both treatment arms (FIG. 16C, Table 2, Table 3). The frequency of CD11b⁺Ly6C⁺Ly6G⁻ cells was significantly increased in the kidney of laquinimod-treated mice (Table 2). CD4 T⁺ cells, CD4⁺Foxp3⁺ Treg, CD19⁺ B cells, and CD11c⁺CD11b⁻ lymphoid-like dendritic cells in the spleen were not affected by laquinimod treatment (Table 3). In contrast, significantly lower frequencies of CD4⁺, CD8⁺ and CD19⁺ cells were observed in the kidneys of PU^{lo} laquinimod- and MMF-treated animals versus controls (Table 2). Laquinimod treatment also resulted in significantly lower CD8⁺ T cells and CD19⁺ B cells versus MMF in PU^{lo} kidneys (Table 2).

### LAQ Induces Expansion of Myeloid-Derived Suppressor Cells

Previous studies have shown that cells co-expressing CD11b, Ly6G and/or Ly6C are endowed with suppressive activity and are called myeloid-derived suppressor cells (MDSC) (13-15). MDSC can be subdivided into two distinct sub-populations with monocytic or granulocytic morphology, defined as CD11b⁺Ly6C⁺Ly6G^{-/low} (mo-MDSC) or CD11b⁺Ly6C^{low}Ly6G+ (gr-MDSC), respectively (13, 15). Because increased frequency of both sub-populations in laquinimod-treated animals was observed, it was further investigated if they possess suppressive function. To address this, CD11b⁺Ly6C⁺Ly6G⁺CD3⁻CD19⁻ or CD11b⁺Ly6C⁺Ly6G⁻CD3⁻CD19⁻ cells sorted from spleens of laquinimod-, MMF- or vehicle-treated animals were co-cultured with anti-CD 3/28 stimulated, CFSE-labeled CD4+ T cells. Both CD11b ly6C⁺Ly6G⁺ and CD11b⁺Ly6C⁺Ly6G⁻ subsets strongly suppressed T cell proliferation (FIG. 16D). Thus, based in the morphology and suppressive activity of these cells, it was concluded that laquinimod induces expansion of gr-MDSC and mo-MDSC. In addition, suppressive function was not dependent on disease activity because gr-MDSC and mo-MDSC isolated from all treatment groups, including water-treated mice, displayed similar suppressive activity (FIGS. 16B, 16C). These data suggest laquinimod might be effective in treating lupus nephritis due in part to its ability to increase quantitative numbers of functional MDSC.

TABLE 2
Leukocyte subsets present in kidney after preventive and therapeutic (PU^{lo}) treatment.

| | Frequency of cells ± SEM | | | | | |
|---|---|---|---|---|---|---|
| | Preventive | | | Therapeutic | | |
| Leukocyte subset | Vehicle | LAQ | MMF | Vehicle | LAQ | MMF |
| CD11b⁺MDSC− | 44.7 ± 0.7 | 31.0 ± 1.8***††† | 49.8 ± 0.7 | 44.7 ± 0.7 | 28.4 ± 0.9***††† | 45.7 ± 0.9 |
| (Mφ) | | | | | | |
| CD11c⁺CD11b+ | 1.4 ± 0.2 | 0.7 ± 0.1**† | 1.0 ± 0.1 | 1.5 ± 0.1 | 0.7 ± 0.1*** | 0.6 ± 0.0*** |
| (DC) | | | | | | |
| CD11c⁺CD11b− | 3.8 ± 0.3 | 3.8 ± 0.2 | 4.4 ± 0.3 | 1.8 ± 0.3 | 1.2 ± 0.1 | 1.3 ± 0.1 |
| (DC) | | | | | | |
| CD11b⁺Ly6C⁺Ly6G− | 21.0 ± 0.9 | 44.4 ± 1.5***††† | 22.9 ± 0.8 | 21.5 ± 0.7 | 41.2 ± 0.4***††† | 22.4 ± 0.3 |
| CD11b⁺Ly6C⁺Ly6G+ | 1.2 ± 0.2 | 7.9 ± 1.9**†† | 1.6 ± 0.2 | 1.3 ± 0.1 | 13.6 ± 1.2***††† | 1.9 ± 0.3 |
| CD4+ | 18.3 ± 0.5 | 7.0 ± 0.5***††† | 19.4 ± 0.4 | 4.2 ± 0.1 | 2.8 ± 0.3***††† | 1.0 ± 0.1*** |
| CD8+ | 9.3 ± 0.3 | 4.2 ± 0.2***††† | 8.8 ± 0.1 | 13.4 ± 0.8 | 2.7 ± 0.2***†† | 6.6 ± 0.2*** |
| CD19+ | 5.7 ± 0.1 | 4.0 ± 0.5**†† | 2.3 ± 0.2*** | 12.2 ± 0.2 | 2.7 ± 0.2***††† | 7.2 ± 0.4*** |

Preventive: 25 weeks after treatment (n = 5-10/group).
Therapeutic (PU^{lo}): 10 wks after initiation of therapy (n = 4-10)
*p < 0.05, **p < 0.01, ***p < 0.001 treatment vs. vehicle;
†p < 0.05, ††p < 0.01, †††p < 0.001 MMF vs. LAQ (1 way ANOVA).

TABLE 3
Leukocyte subsets present in spleen after preventive and therapeutic (PU^{lo}) treatment.

| | Frequency of cells ± SEM | | | | | |
|---|---|---|---|---|---|---|
| | Preventive | | | Therapeutic | | |
| Leukocyte subset | Vehicle | LAQ | MMF | Vehicle | LAQ | MMF |
| CD11b⁺MDSC−(Mφ) | 55.8 ± 3.2 | 25.2 ± 1.7***††† | 59.3 ± 2.3 | 45.3 ± 2.9 | 25.6 ± 3.3**† | 40.5 ± 3.1 |
| CD11c⁺CD11b+(DC) | 1.4 ± 0.2 | 0.7 ± 0.1**†† | 1.0 ± 0.1 | 1.4 ± 0.2 | 1.0 ± 0.2 | 0.9 ± 0.2 |
| CD11c⁺CD11b− (DC) | 3.8 ± 0.3 | 3.8 ± 0.2 | 4.4 ± 0.3 | 5.1 ± 0.6 | 4.5 ± 0.4 | 4.1 ± 0.7 |
| CD11b⁺Ly6C⁺Ly6G− | 37.7 ± 6.2 | 37.5 ± 2.1 | 33.4 ± 1.8 | 31.3 ± 3.3 | 38.7 ± 1.9 | 30.4 ± 2.7 |
| CD11b⁺Ly6C⁺Ly6G+ | 12.9 ± 2.3 | 31.7 ± 3.6**†† | 15. ± 1.6 | 16.6 ± 2.6 | 33.2 ± 5.6*† | 17.5 ± 2.4 |
| CD4+ | 22.5 ± 1.1 | 20.2 ± 0.5 | 26.2 ± 1.3 | 23.4 ± 0.8 | 21.3 ± 1.3 | 20.1 ± 0.7 |
| CD8+ | 8.9 ± 1.0 | 12.3 ± 0.7* | 12.4 ± 0.9 | 17.1 ± 3.6 | 24.9 ± 2.9† | 13.5 ± 2.1 |
| CD4⁺CD25⁺Foxp3⁺ | 8.9 ± 0.8 | 9.4 ± 0.6 | 7.9 ± 0.4 | 3.7 ± 0.5 | 2.2 ± 0.1 | 3.2 ± 0.8 |
| CD19+ | 65.5 ± 4.6 | 68.0 ± 3.8 | 57.4 ± 3.9 | 45.1 ± 3.5 | 51.1 ± 3.3 | 45.4 ± 3.3 |

Preventive: 25 weeks after treatment (n = 5-10/group).
Therapeutic (PU^{lo}): 10 wks after initiation of therapy (n = 4-10)
*p < 0.05, **p < 0.01, ***p < 0.001 treatment vs. vehicle;
†p < 0.05, ††p < 0.01, †††p < 0.001 MMF vs. LAQ (1 way ANOVA).

### LAQ Promotes Cytokine Shift to Anti-Inflammatory Profile in BWF1 Mice

Recent evidence suggests that laquinimod induces anti-inflammatory type II monocytes in EAE (8, 12). Therefore, cytokine production by splenic M/M (CD11b⁺Ly6C⁻Ly6G⁻) after stimulation with TLR agonists was investigated.

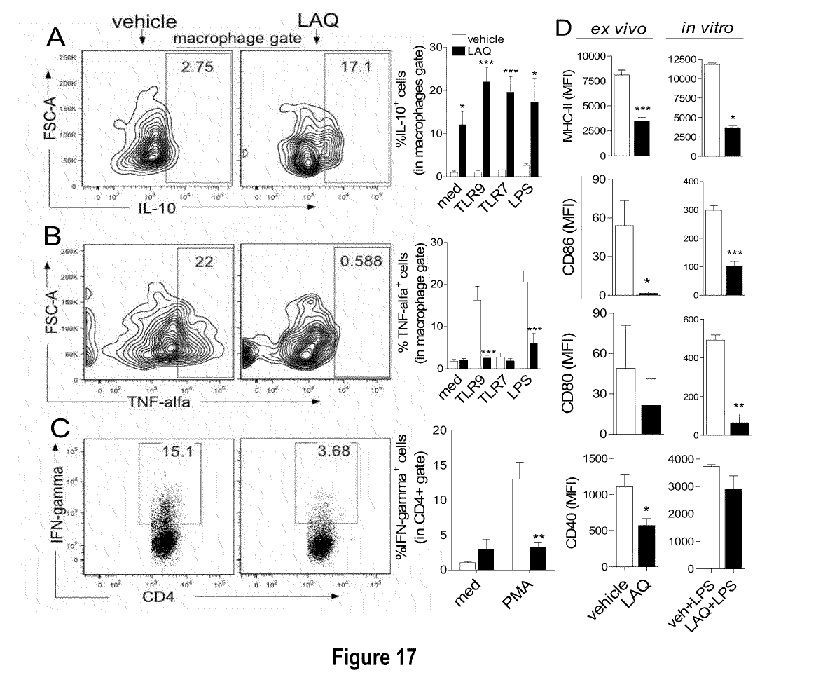

Significantly more M/M from laquinimod-treated mice were IL-10⁺ than vehicle-treated cells (FIG. 17A). In contrast, significantly fewer M/M and CD4⁺ T cells from laquinimod-treated animals produced pro-inflammatory TNF-α (FIG. 17B) or IFN-γ (FIG. 17C), respectively, versus M/M isolated from vehicle-treated mice. A detailed ex vivo surface phenotypic analysis of M/M from laquinimod- and vehicle-treated animals revealed decreased expression of MHC 11, CD86 and CD40 (FIG. 17D). Similar down-regulation of these molecules was observed on dendritic cells (CD11c⁺) (data not shown). Expression of MHC 11, CD86 and CD80 was significantly lower on M/M isolated from laquinimod-treated mice even after in vitro stimulation with LPS (1 μg/mL) (FIG. 17D). Ex vivo and in vitro expression of MHC II on B cells (CD19+) was also significantly down-regulated (data not shown). These results suggest that putative mechanisms of laquinimod action in lupus nephritis may involve a switch from pro-inflammatory (type I) to anti-inflammatory (type II) M/M, inhibition of pro-inflammatory Th1 cells by up-regulated MDSCs, and down-regulation of activation/co-stimulatory molecules on antigen-presenting cells.

### Discussion

The immunomodulatory properties of laquinimod in murine lupus nephritis were explored. The data indicate that laquinimod treatment, whether preventive or therapeutic, led to prevention or improvement of established proteinuria, decreased rise in serum creatinine, and improved survival. In mice with active advanced disease as defined by the presence of anti-dsDNA and proteinuria ≧300 mg/dL, laquinimod significantly improved survival and proteinuria, which MMF treatment failed to do. In addition, inhibition of clinical disease by laquinimod associated with increased MDSC numbers, a switch of M/M from pro-inflammatory type I to anti-inflammatory type II M/M, and reduced pro-inflammatory Th1 cells, which in turn modulates immune responses in vivo. MDSC and M/M numbers which decreased in laquinimod-treated mice were unchanged in MMF-treated mice. Notably, modulation of these cell types is observed in not only in the spleen, but also in the kidney of laquinimod-treated mice, the target tissue in lupus nephritis, and may represent a distinct advantage of laquinimod therapy over commonly used therapeutics when used in human SLE.

The major cause of death in BWF1 mice is glomerulonephritis (16). Thus, it would be expected that improved survival and prevention of rise in creatinine in laquinimod-treated animals correlate with protection from kidney damage. Histologic analysis of preventive and therapeutic treatments demonstrated laquinimod significantly reduced histological scores, including endocapillary hypercellularity and FSGS that indicate inflammation in the glomeruli, and supports the assumption that laquinimod protects from kidney disease. However, the increase in survival and stabilized serum creatinine observed in PU^{hi} mice was not associated with healing of chronic changes on renal histology. This could be because damage was well established and irreversible in mice with advanced disease. In addition, the persistence of high levels of anti-dsDNA in laquinimod/MMF-treated after disease was established might have contributed to continuing damage. Dissociation between anti-DNA antibodies and improvement in glomerulonephritis has been reported in other studies (17, 18).

In previous studies, it was observed that the beneficial effect of laquinimod in EAE models was associated with a cytokine shift from pro-inflammatory to an anti-inflammatory M/M phenotype (7, 12, 19). Most recently, it was reported that laquinimod treatment induced anti-inflammatory monocytes (type II) and reduced secretion of IFNγ and IL-17 in EAE (8, 12, 20). Similarly, it has been demonstrated that laquinimod contributes to immune modulation in lupus nephritis by down-regulation of activation/co-stimulatory molecules and induction of type II monocyte/macrophages, or possibly a switch from type I to type II in resident macrophages. Inflammatory/anti-inflammatory functions of monocytes/macrophages exist along a spectrum. The studies were simplified by defining pro-inflammatory, type I M/M as secreting TNFα and/or IFNγ, and displaying high quantities of MHC Class II, CD86, CD80, and CD40. Anti-inflammatory type II M/M were defined as IL-10 secreting and lower expression levels of the surface molecules mentioned above. Laquinimod treatment also led to reduced frequencies of pro-inflammatory IFNγ⁺CD4⁺ T cells. Whether this is a direct or indirect effect on M/M T cells requires further investigation, although a previous report supports a more indirect impact of laquinimod on T cells through antigen presenting cells in EAE (8). In addition, laquinimod reduced numbers of T (CD4⁺ and CD8⁺) and B (CD19+) cells in the kidneys, as well as M/M. As expected, MMF altered kidney infiltration of B and T lymphocytes, although MMF did not affect M/M or MDSC numbers. Previous studies have suggested that laquinimod exerts beneficial effects in EAE through inhibition of leukocyte migration into the central nervous system (7, 19, 21, 22). In the study, inhibition of leukocyte infiltration with concomitant increased MDSC infiltration into the kidneys after laquinimod treatment correlate with reduced kidney damage (in PU^{lo} mice) and is a potential mechanism for laquinimod protection of lupus nephritis.

Both preventive and therapeutic laquinimod treatment induced expansion of two major subsets of myeloid-derived suppressor cells: granulocytic-MDSC (CD11b⁺Ly6G⁺Ly6C⁺) and monocytic-MDSC(CD11b⁺Ly6G⁻Ly6C⁺) in BWF1 mice. MDSC consist of a heterogeneous population of immature myeloid cells, immature granulocytes, monocytes-macrophages, dendritic cells and myeloid progenitor cells (23). In mice, they are defined as CD11b⁺Gr1⁺ cells. Gr1 is an antibody (RB6-8C5) that detects both Ly6G, a molecule expressed on granulocytes (24), and Ly6C, a molecule highly expressed on monocytes (25, 26). MDSC were originally described more than 25 years ago in patients with cancer (27) and have become the focus of intense study for immunologists in recent years after studies by Bronte and colleagues (28). MDSC are believed to regulate immune response in many pathologic conditions, including infections (29-31), acute inflammation (32), and different autoimmune diseases such as encephalomyelitis (33), colitis (34), diabetes (35), and a murine model of rheumatoid arthritis (36). There are no known reports describing their role in lupus nephritis. Described here are the expansion of both gr-MDSC and mo-MDSC in a murine model of lupus nephritis in response to treatment with laquinimod. Their suppressive activity appears to be independent of therapeutic regimen, suggesting that the effect of laquinimod on MDSC is most likely quantitative rather than qualitative. A therapeutic option that increases regulatory cells may be of interest in lupus nephritis. It was also observed a trend toward more potent suppressive capacity by mo-MDSC than gr-MDSC, in agreement with previous studies (23, 24). In addition, mo-MDSCs were expanded in kidneys but not spleens of laquinimod-treated animals. It was found that production of arginase and reactive oxygen species, two of the major factors involved in MDSC immune suppression, were not affected by laquinimod treatment (data not shown) (23). The importance of MDSC expansion in the target organ and the role of these regulatory cells in the prevention of kidney pathology, therefore, require further investigation. A recent report also suggests that Treg are increased in the CNS of EAE mice (37). Treg numbers in kidney were not assessed in the study, and although splenic Treg seemed to be unchanged in spleen

A phase 3 clinical trial of laquinimod in multiple sclerosis demonstrated that the adverse events profile of laquinimod is mild, so the drug might also be safe in humans with lupus nephritis. Laquinimod-treated MS patients had a 2.6 higher risk for elevated alanine aminotransferase levels, 2.0 higher risk for abdominal pain, and 1.8 higher risk for back pain (9). This is a much more favorable side effects profile than both MMF (gastrointestinal distress, infections, leukopenia) and roquinimex, a quinolone closely related to laquinimod that was pulled out of a clinical trial because of serious cardiovascular toxicities (38). A recent trial of laquinimod in human lupus nephritis has been fully enrolled and preliminary results will be presented soon (clinicaltrials.gov, searched June 2013). It is noteworthy that the laquinimod dosage used in these studies is similar to the dosage in the MS clinical trial when adjusting for body surface area differences between humans and mice (39). In addition, paquinimod, a related quinoline to laquinimod that differs at a single side chain (a chloride ion in laquinimod versus an ethyl group in paquinimod) also showed a favorable adverse events profile in human lupus in a recent phase 1b trial and was effective at treating a different murine SLE model (40).

The findings suggest that laquinimod ameliorates murine lupus nephritis through multiple mechanisms. Numbers of pro-inflammatory T and B lymphocytes and M/M were reduced in the target tissue (kidney). In addition, a shift from pro-inflammatory type I to anti-inflammatory type II M/M occurred. Finally, two types of myeloid suppressor cells were induced. Laquinimod treatment was significantly better than MMF with regard to survival and reduction of proteinuria in mice with advanced lupus nephritis. Laquinimod is a promising immunomodulatory therapeutic for use in human SLE and could be useful in treating human lupus, where it is currently in clinical development.

References cited in the example include the following:
1) Hahn B H. The pathogenesis of SLE. In: Wallace D J and Hahn B H editors. Dubois' Lupus Erythematosus and Related Syndromes. Philadelphia: Elsevier Saunders; 2013. pp. 25-34.
2) Davidson A, Aranow C. Lupus nephritis: lessons from murine models. Nat Rev Rheumatol 2010; 6:13-20.
3) Hahn B H, McMahon M A, Wilkinson A, Wallace W D, Daikh D I, Fitzgerald J D, et al. American College of Rheumatology guidelines for screening, treatment, and management of lupus nephritis. Arthritis Care Res (Hoboken) 2012; 64:797-808.
4) Schiffer L, Bethunaickan R, Ramanujam M, Huang W, Schiffer M, Tao H, et al. Activated renal macrophages are markers of disease onset and disease remission in lupus nephritis. J Immunol 2008; 180:1938-1947.
5) Yang N, Isbel N M, Nikolic-Paterson D J, Li Y, Ye R, Atkins R C, et al. Local macrophage proliferation in human glomerulonephritis. Kidney Int 1998; 54:143-151.
6) Hoi A Y, Hickey M J, Hall P, Yamana J, O'Sullivan K M, Santos L L, et al. Macrophage migration inhibitory factor deficiency attenuates macrophage recruitment, glomerulonephritis, and lethality in MRL/lpr mice. J Immunol 2006; 177:5687-5696.
7) Wegner C, Stadelmann C, Pfortner R, Raymond E, Feigelson S, Alon R, et al. Laquinimod interferes with migratory capacity of T cells and reduces IL-17 levels, inflammatory demyelination and acute axonal damage in mice with experimental autoimmune encephalomyelitis. J Neuroimmunol 2010; 227:133-143.
8) Schulze-Topphoff U, Shetty A, Varrin-Doyer M, Molnarfi N, Sagan S A,
Sobel R A, et al. Laquinimod, a quinoline-3-carboxamide, induces type II myeloid cells that modulate central nervous system autoimmunity. PLoS ONE 2012; 7:e33797.
9) Comi G, Jeffery D, Kappos L, Montalban X, Boyko A, Rocca M A, et al.
Placebo-controlled trial of oral laquinimod for multiple sclerosis. N Engl J Med 2012; 366:1000-1009.
10) Skaggs B J, Hahn B H, Sahakian L, Grossman J, McMahon M. Dysfunctional, pro-inflammatory HDL directly upregulates monocyte PDGFRbeta, chemotaxis and TNFalpha production. Clin Immunol 2010; 137:147-156.
11) Hahn B, Kono D. Animal models of SLE. In: Wallace D and Hahn B editors. Dubois' Lupus Erythematosus and Related Syndromes. Philadelphia: Elsevier Sounders; 2013. pp. 190-236.
12) Thone J, Ellrichmann G, Seubert S, Peruga I, Lee D H, Conrad R, et al. Modulation of autoimmune demyelination by laquinimod via induction of brain-derived neurotrophic factor. Am J Pathol 2012; 180:267-274.
13) Youn J I, Nagaraj S, Collazo M, Gabrilovich D I. Subsets of myeloid-derived suppressor cells in tumor-bearing mice. J Immunol 2008; 181:5791-5802.
14) Peranzoni E, Zilio S, Marigo I, Dolcetti L, Zanovello P, Mandruzzato S, et al. Myeloid-derived suppressor cell heterogeneity and subset definition. Curr Opin Immunol 2010; 22:238-244.
15) Movahedi K, Guilliams M, Van den Bossche J, Van den Bergh R, Gysemans C, Beschin A, et al. Identification of discrete tumor-induced myeloid-derived suppressor cell subpopulations with distinct T cell-suppressive activity. Blood 2008; 111:4233-4244.
16) Howie J B, Helyer B J. The immunology and pathology of NZB mice. Advances in immunology 1968; 9:215-266.
17) Zurier R B, Sayadoff D M, Torrey A B, Rothfield N F. Prostaglandin E treatment of NZB/NZW mice. Arthritis Rheum 1977; 20:723-728.
18) Tarkowski A, Gunnarsson K, Stalhandske T. Effects of LS-2616 administration upon the autoimmune disease of (NZB×NZW) F1 hybrid mice. Immunology 1986; 59:589-594.
19) Bruck W, Wegner C. Insight into the mechanism of laquinimod action. J Neurol Sci 2011; 306:173-179.
20) Weber M S, Prod'homme T, Youssef S, Dunn S E, Rundle C D, Lee L, et al. Type II monocytes modulate T cell-mediated central nervous system autoimmune disease. Nat Med 2007; 13:935-943.
21) Mishra M K, Wang J, Silva C, Mack M, Yong V W. Kinetics of proinflammatory monocytes in a model of multiple sclerosis and its perturbation by laquinimod. Am J Pathol 2012; 181:642-651.
22) Brunmark C, Runstrom A, Ohlsson L, Sparre B, Brodin T, Astrom M, et al. The new orally active immunoregulator laquinimod (ABR-215062) effectively inhibits development and relapses of experimental autoimmune encephalomyelitis. J Neuroimmunol 2002; 130:163-172.
23) Youn J I, Gabrilovich D I. The biology of myeloid-derived suppressor cells: the blessing and the curse of morphological and functional heterogeneity. Eur J Immunol 2010; 40:2969-2975.
24) Fleming T J, Fleming M L, Malek T R. Selective expression of Ly-6G on myeloid lineage cells in mouse bone marrow. RB6-8C5 mAb to granulocyte-differentiation antigen (Gr-1) detects members of the Ly-6 family. J Immunol 1993; 151:2399-2408.
25) Sunderkotter C, Nikolic T, Dillon M J, Van Rooijen N, Stehling M, Drevets D A, et al. Subpopulations of mouse blood monocytes differ in maturation stage and inflammatory response. J Immunol 2004; 172:4410-4417.
26) Greten T F, Manns M P, Korangy F. Myeloid derived suppressor cells in human diseases. Int Immunopharmacol 2011; 11:802-807.
27) Young M R, Newby M, Wepsic H T. Hematopoiesis and suppressor bone marrow cells in mice bearing large metastatic Lewis lung carcinoma tumors. Cancer Res 1987; 47:100-105.
28) Bronte V, Wang M, Overwijk W W, Surman D R, Pericle F, Rosenberg S A, et al. Apoptotic death of CD8+ T lymphocytes after immunization: induction of a suppressive population of Mac-1+/Gr-1+ cells. J Immunol 1998; 161:5313-5320.
29) Gabrilovich D I, Nagaraj S. Myeloid-derived suppressor cells as regulators of the immune system. Nat Rev Immunol 2009; 9:162-174.
30) Bowen J L, Olson J K Innate immune CD11b+Gr-1+ cells, suppressor cells, affect the immune response during Theiler's virus-induced demyelinating disease. J Immunol 2009; 183:6971-6980.
31) Van Ginderachter J A, Beschin A, De Baetselier P, Raes G. Myeloid-derived suppressor cells in parasitic infections. Eur J Immunol 2010; 40:2976-2985.
32) Greifenberg V, Ribechini E, Rossner S, Lutz M B. Myeloid-derived suppressor cell activation by combined LPS and IFN-gamma treatment impairs DC development. Eur J Immuno12009; 39:2865-2876.
33) Zhu B, Bando Y, Xiao S, Yang K, Anderson A C, Kuchroo V K, et al. CD11b+Ly-6C(hi) suppressive monocytes in experimental autoimmune encephalomyelitis. J Immuno12007; 179:5228-5237.
34) Haile L A, von Wasielewski R, Gamrekelashvili J, Kruger C, Bachmann 0, Westendorf A M, et al. Myeloid-derived suppressor cells in inflammatory bowel disease: a new immunoregulatory pathway. Gastroenterology 2008; 135:871-881, 881 e871-875.
35) Yin B, Ma G, Yen C Y, Zhou Z, Wang G X, Divino C M, et al. Myeloid-derived suppressor cells prevent type 1 diabetes in murine models. J Immunol 2010; 185:5828-5834.
36) Charles J F, Hsu L Y, Niemi E C, Weiss A, Aliprantis A O, Nakamura M C. Inflammatory arthritis increases mouse osteoclast precursors with myeloid suppressor function. J Clin Invest 2012; 122:4592-4605.
37) Aharoni R, Saada R, Eilam R, Hayardeny L, Sela M, Amon R. Oral treatment with laquinimod augments regulatory T-cells and brain-derived neurotrophic factor expression and reduces injury in the CNS of mice with experimental autoimmune encephalomyelitis. J Neuroimmunol 2012; 251(1-2):14-24. Epub 2012 Jun. 28.
38) Noseworthy J H, Wolinsky J S, Lublin F D, Whitaker J N, Linde A, Gjorstrup P, et al. Linomide in relapsing and secondary progressive MS: part I: trial design and clinical results. North American Linomide Investigators. Neurology 2000; 54:1726-1733.
39) Reagan-Shaw S, Nihal M, Ahmad N. Dose translation from animal to human studies revisited. Faseb J 2008; 22:659-661.
40) Bengtsson A A, Sturfelt G, Lood C, Ronnblom L, van Vollenhoven R F, Axelsson B, et al. Pharmacokinetics, tolerability, and preliminary efficacy of paquinimod (ABR-215757), a new quinoline-3-carboxamide derivative: studies in lupus-prone mice and a multicenter, randomized, double-blind, placebo-controlled, repeat-dose, dose-ranging study in patients with systemic lupus erythematosus. Arthritis Rheum 2012; 64:1579-1588.
While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety. Throughout this application, various publications are referred to by first author and year of publication. Full citations for these publications are presented in a References section immediately before the claims. Disclosures of the publications cited in the References section in their entireties are hereby incorporated by reference into this application in order to more fully describe the state of the art as of the date of the invention described herein. In closing, it is to be understood that the embodiments of the invention disclosed herein are illustrative of the principles. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A method for delaying or preventing onset of active lupus nephritis in a mammal at risk for developing active lupus nephritis, comprising:
periodically administering to the mammal an amount of laquinimod effective to delay onset of active lupus nephritis in the mammal.

2. The method of claim 1, wherein the mammal is afflicted with class I lupus nephritis.

3. The method of claim 1, wherein the mammal is afflicted with class II lupus nephritis.

4. The method of claim 1, wherein the mammal's protein to creatinine ratio at baseline is less than 3.

5. The method of claim 4, wherein the mammal's protein to creatinine ratio at baseline is less than 2.

6. The method of claim 5, wherein the mammal's protein to creatinine ratio at baseline is less than 1.

7. The method of claim 1, wherein the laquinimod is a pharmaceutically acceptable salt of laquinimod.

8. The method of claim 7, wherein the pharmaceutically acceptable salt of laquinimod is laquinimod sodium.

9. The method of claim 1, wherein the periodic administration of laquinimod is effected orally.

10. The method of claim 1, wherein the amount of laquinimod is selected from the group consisting of 0.25-2.0 mg/day, 0.25 mg/day, 0.3 mg/day, 0.5 mg/day, 1.5 mg/day, 0.5-1.2 mg/day, 0.6 mg/day, 1.0 mg/day, and 1.2 mg/day.

11. The method of claim 1, wherein the amount of laquinimod is effective to prevent onset of active lupus nephritis in the mammal.

12. The method of claim 1, wherein the amount of laquinimod is effective to delay or prevent a symptom of active lupus nephritis in the mammal.

13. The method of claim 12, wherein the symptom is selected from the group consisting of proteinuria, an increase of protein to creatinine ratio, an increase of immune complex deposition, serum anti-DNA antibody production, edema in the mammal, and hypertension in the mammal.

14. The method of claim 12, wherein the amount of laquinimod is effective to delay or prevent increase of glomerular immunoglobulin deposition in the mammal.

15. The method of claim 14, wherein the amount of laquinimod is effective to delay or prevent increase of glomerular Complement component 3 (C3) deposition in the mammal.

16. The method of claim 14, wherein the amount of laquinimod is effective to delay or prevent increase of glomerular Complement component 3 (C3) deposition in the mammal.

17. The method of claim 1, wherein the amount of laquinimod is effective to reduce the mammal's protein to creatinine ratio.

18. The method of claim 17, wherein the mammal's protein to creatinine ratio is reduced by at least 50% as compared to baseline.

19. The method of claim 17, wherein the mammal's protein to creatinine ratio is reduced to no more than 0.3.

20. The method of claim 18, wherein the mammal's protein to creatinine ratio is reduced to no more than 0.3.

21. The method of claim 1, wherein the periodic administration continues for at least 24 weeks.

22. The method of claim 1, wherein the mammal is human.

23. Laquinimod for use in delaying onset of active lupus nephritis in a mammal at risk for developing active lupus nephritis.

24. A method for treating or alleviating a symptom associated with active lupus nephritis in a mammal diagnosed with active lupus nephritis, comprising:
periodically administering to the mammal an amount of laquinimod effective to treat or alleviate a symptom associated with active lupus nephritis in the mammal.

25. The method of claim 24, wherein the symptom is selected from a group consisting of elevated creatine level, proteinuria, hematuria, red blood cell casts, granular casts, microhematuria, macrohematuria, reduced renal function; rapidly progressive glomerulonephritis, acute renal failure, hyperkalemia; hypertension, tubular abnormalities; uremia due to retention of waste products and renal insufficiency such as azotemia (elevated blood nitrogen) and oliguria (low urine output <400 mL/day), a malar rash, a discoid rash, a photosensitivity, an oral ulcer, a nonerosive arthritis, a pleuropericarditis, and neurological manifestations, and hematological disorders.

26. A pharmaceutical composition comprising:
an amount of laquinimod for use in delaying onset of active lupus nephritis in a mammal at risk for developing active lupus nephritis, and
a pharmaceutical carrier or adjuvant.

27. A composition comprising an active ingredient or compound which is effective for: induction of at least two types of regulatory cells that can suppress autoimmunity, and reduction of numbers of circulating moncytes/macrophages.

28. The composition of claim 27, wherein the compound is Laquinimod (LAQ).

29. The composition of claim 27, wherein the compound is effective for lupus nephritis.

30. The composition of claim 27, wherein the regulatory cells express one or more markers selected from the group consisting of CD3, CD4, CD8, CD11, CD19, CD25, CD28, Foxp3, Tr1, Th3, Qa-1, Ly6G and Ly6C.

31. The composition of claim 27, further comprising a pharmaceutically acceptable carrier.

32. The composition of any of claim 32, which is an oral formulation.

33. A method of treating, ameliorating, or preventing lupus nephritis, comprising:
applying to a mammal a composition according to any of claims 27.
